(19) United States
(12) Patent Application Publication
(10) Pub. No.: US 2018/0288404 A1
(43) Pub. Date: Oct. 4, 2018

(54) IMAGE PROCESSING SYSTEM, IMAGE PROCESSING APPARATUS, PROJECTING APPARATUS, AND PROJECTION METHOD

(71) Applicants: NATIONAL INSTITUTE OF ADVANCED INDUSTRIAL SCIENCE AND TECHNOLOGY, Tokyo (JP); NIKON CORPORATION, Tokyo (JP)

(72) Inventors: Yuzuru IKEHARA, Tsukuba-shi (JP); Susumu Makinouchi, Yokohama-shi (JP); Tetsuro Ishikawa, Osaka (JP)

(73) Assignees: NATIONAL INSTITUTE OF ADVANCED INDUSTRIAL SCIENCE AND TECHNOLOGY, Tokyo (JP); NIKON CORPORATION, Tokyo (JP)

(21) Appl. No.: 15/917,004

(22) Filed: Mar. 9, 2018

Related U.S. Application Data
(63) Continuation of application No. PCT/JP2016/076329, filed on Sep. 7, 2016.

(30) Foreign Application Priority Data
Sep. 11, 2015 (JP) 2015-179516

(51) Int. Cl. H04N 13/341; H04N 5/33; H04N 13/243; H04N 13/344; H04N 13/211; H04N 13/346; A61B 5/00; G02B 27/01

(52) U.S. Cl. CPC H04N 13/341; H04N 5/33; H04N 13/243; H04N 13/344; H04N 13/211; H04N 13/346; G02B 2027/014; A61B 5/4887; A61B 5/743; A61B 5/7445; G02B 27/0172; G02B 2027/0178; G02B 2027/0141; A61B 5/0077

(57) ABSTRACT

A technique that only and simply projects a video of a specific region on a body tissue is insufficient in some cases as an assistance technique for users in, for example, surgeries and pathological examinations. An image processing system includes an infrared light irradiating apparatus, an optical detector, a control apparatus, a display apparatus, and a projecting apparatus. The infrared light irradiating apparatus is configured to irradiate a biological tissue with infrared light. The optical detector is configured to detect light radiated from the biological tissue irradiated with the infrared light. The control apparatus is configured to create an image of the biological tissue using a detection result by the optical detector. The display apparatus is configured to display the created image. The projecting apparatus is configured to irradiate the biological tissue with first light. The control apparatus is configured to control the irradiation with the first light by the projecting apparatus such that contents of an input are reflected to the biological tissue in response to the input to the display apparatus configured to display the image of the biological tissue (see FIG. 1).

### CROSS REFERENCE TO RELATED APPLICATIONS

This is a Continuation of PCT International Application PCT/JP2016/076329 filed on Mar. 16, 2017, which in turns claims benefit of Japanese patent application 2015-179516 filed in Japan on Sep. 11, 2015. The entire contents of each of the above documents is hereby incorporated by reference into the present application.

### TECHNICAL FIELD

The present invention relates to an image processing system, an image processing apparatus, a projecting apparatus, and a projection method.

### BACKGROUND

In a field such as a medical treatment, there has been proposed a technique to project an image on a tissue (for example, see the following Patent Literature 1). For example, an apparatus according to Patent Literature 1 irradiates a body tissue with infrared to obtain a video of a subdermal vessel based on the infrared reflected by the body tissue. This apparatus projects a visible optical image of the subdermal vessel on a surface of the body tissue. Thus, doctors and nurses can visibly confirm even a blood vessel hard to be visually recognized at a part to which an injection is to be punctured, thereby ensuring the injection by an operation with both hands.

However, like Patent Literature 1, for example, the technique that only and simply projects the video of a specific region on the body tissue is insufficient in some cases as an assistance technique for users (for example, doctors and laboratory technicians) in, for example, surgeries and pathological examinations.

### PATENT LITERATURE

Patent Literature 1: JP 2006-102360 A

### SUMMARY

According to a first embodiment, there is provided an image processing system that includes an infrared light irradiating apparatus, an optical detector, a display apparatus, a projecting apparatus, and a control apparatus. The infrared light irradiating apparatus is configured to irradiate a biological tissue with infrared light. The optical detector is configured to detect detection light radiated from the biological tissue irradiated with the infrared light. The display apparatus is configured to display an image of the biological tissue. The image being created using a detection result by the optical detector. The projecting apparatus is configured to irradiate the biological tissue with first light. The control apparatus is configured to control the irradiation with the first light by the projecting apparatus such that contents of an input are reflected to the biological tissue based on the input to the display apparatus configured to display the image of the biological tissue.

According to a second embodiment, there is provided an image processing system that includes an infrared light irradiating apparatus, an optical detector, a display apparatus, a projecting apparatus, and a control apparatus. The infrared light irradiating apparatus is configured to irradiate a biological tissue with infrared light. The optical detector is configured to detect detection light radiated from the biological tissue irradiated with the infrared light. The display apparatus is configured to display an image of the biological tissue. The image is created using a detection result by the optical detector. The projecting apparatus is configured to irradiate the biological tissue with light. The control apparatus is configured to analyze the detection result by the optical detector to identify an affected part in the biological tissue. The control apparatus is configured to superimpose information on the affected part on the image of the biological tissue and cause the display apparatus to display the superimposed image. The control apparatus is configured to control the irradiation of the information on the affected part to the biological tissue by the projecting apparatus.

According to a third embodiment, there is provided an image processing system that includes a controller. The controller is configured to create an image of a biological tissue using a detection result by an optical detector. The optical detector is configured to detect detection light radiated from the biological tissue irradiated with infrared light. The controller is configured to transmit the created image to a display apparatus such that the display apparatus displays the created image. The controller is configured to control an irradiation by a projector such that the projector irradiates the biological tissue with light to reflect contents of an input to the biological tissue based on the input to the display apparatus configured to display the image of the biological tissue.

According to a fourth embodiment, there is provided an image processing apparatus that includes a controller. The controller is configured to create an image of a biological tissue using a detection result by an optical detector. The optical detector is configured to detect detection light radiated from the biological tissue irradiated with infrared light. The controller is configured to transmit the created image to a display apparatus such that the display apparatus displays the created image. The controller is configured to analyze the detection result by the optical detector to identify an affected part in the biological tissue. The controller is configured to superimpose information on the affected part on the image of the biological tissue and cause the display apparatus to display the superimposed image. The controller is configured to control an irradiation of the information on the affected part to the biological tissue by a projector configured to irradiate the biological tissue with light.

According to a fifth embodiment, there is provided a projection method that includes irradiating, detecting, creating, displaying, and controlling. The irradiating irradiates a biological tissue with infrared light. The detecting detects detection light radiated from the biological tissue irradiated with the infrared light. The creating creates an image of the biological tissue using a detection result of the detection light. The displaying displays the image of the biological tissue on a display apparatus. The controlling controls an irradiation of light by a projecting apparatus such that contents of an input are reflected to the biological tissue based on the input to the display apparatus configured to display the image of the biological tissue.

According to a sixth embodiment, there is provided a projection method that includes irradiating, detecting, creating, displaying, analyzing, and controlling. The irradiating irradiates a biological tissue with infrared light. The detecting detects detection light radiated from the biological tissue irradiated with the infrared light. The creating creates an image of the biological tissue using a detection result of the detection light. The displaying displays the image of the biological tissue on a display apparatus. The analyzing analyzes the detection result to identify an affected part in the biological tissue. The analyzing superimposes information on the affected part on the image of the biological tissue and causes the display apparatus to display the superimposed image. The controlling controls an irradiation of the information on the affected part to the biological tissue by a projecting apparatus configured to irradiate the biological tissue with light.

According to a seventh embodiment, there is provided a projecting apparatus that includes a projector and a controller. The projector is configured to irradiate a biological tissue with first light. The controller is configured to control the irradiation with the first light by the projector such that contents of an input are reflected to the biological tissue based on the input to a display apparatus configured to display an image of the biological tissue. The image is created using a detection result by an optical detector. The optical detector is configured to detect detection light radiated from the biological tissue irradiated with infrared light.

According to an eighth embodiment, there is provided a projecting apparatus that includes a projector and a controller. The projector is configured to irradiate a biological tissue with light. The controller is configured to analyze a detection result by an optical detector configured to detect detection light radiated from the biological tissue irradiated with infrared light to identify an affected part in the biological tissue. The controller is configured to superimpose information on the affected part on an image of the biological tissue and cause a display apparatus to display the superimposed image. The controller is configured to control an irradiation of the information on the affected part to the biological tissue by the projector.

### BRIEF DESCRIPTION OF DRAWINGS

FIG. 1 is a drawing illustrating an example of a schematic configuration of an image processing system 1 according to an embodiment.

FIG. 2 is a drawing illustrating an example of a pixel array of an image according to this embodiment.

FIG. 3 is a drawing illustrating an example of a distribution of absorbance in a near-infrared wavelength region according to this embodiment.

FIG. 4 is a flowchart describing process contents in Function 1 according to this embodiment.

FIG. 5 is a drawing describing an overview of a behavior in Function 2 according to this embodiment.

FIG. 6 is a flowchart describing process contents in Function 2 according to this embodiment.

FIG. 7 is a drawing describing an overview of a behavior in Function 3 according to this embodiment.

FIG. 8 is a flowchart describing process contents in Function 3 according to this embodiment.

FIG. 9 is a drawing describing an overview of a behavior in Function 4 according to this embodiment.

FIG. 10 is a flowchart describing process contents in Function 4 according to this embodiment.

FIG. 11 is a drawing describing Function 5 according to this embodiment and a drawing illustrating an example of a schematic configuration of the image processing system 1 used in an operating room.

FIG. 12 is a drawing illustrating an example of a schematic configuration of the image processing system 1 according to this embodiment used in the operating room.

FIG. 13 is a drawing describing an overview of a behavior in Function 6 according to this embodiment.

FIG. 14 is a timing chart illustrating switching timings to open/close liquid crystal shutters on liquid crystal shutter glasses 81, a lighting timing of a surgery shadowless lamp 71, and a timing of guide light irradiation (projection) according to this embodiment.

FIG. 15 is a drawing illustrating a configuration of an irradiator 2 according to a modification of this embodiment.

FIG. 16 is a drawing illustrating a configuration of an optical detector 3 according to the modification of this embodiment.

FIG. 17 is a drawing illustrating a configuration of a projector 5 according to the modification of this embodiment.

FIG. 18 is a drawing illustrating a configuration of the image processing system 1 according to the modification of this embodiment.

FIG. 19 is a timing chart illustrating one example of behaviors of the irradiator 2 and the projector 5 according to the modification of this embodiment.

FIG. 20 is a drawing illustrating an example of a schematic configuration of the image processing system 1 having a function to perform fluorescent observation on a tissue BT of this embodiment.

FIG. 21 is a drawing illustrating an example of a schematic configuration of the image processing system 1 having a function to process a multi-modality image of this embodiment.

### DETAILED DESCRIPTION OF EMBODIMENTS

The following describes embodiments with reference to the accompanying drawings. The accompanying drawings represent functionally identical elements by identical reference numerals in some cases. Although the accompanying drawings illustrate the embodiments and examples of mounting according to a principle of the present invention, these drawings are for understanding of the present invention and never used for limited interpretation of the present invention. The explanations of this description are merely typical examples and therefore do not limit the claims and application examples of the present invention by any means.

While the embodiments give the explanation in detail enough for a person skilled in the art to carry out this invention, it is necessary to understand that other mountings and forms are possible and that changes in configurations and structures and substitutions of various components can be made without departing from the scope and spirit of the technical idea of this invention. Therefore, the following description should not be interpreted to be limited.

Further, as described later, the embodiments may be mounted by software running on a general-purpose computer, by dedicated hardware, or by a combination of software and hardware.

<Configuration of Image Processing System>

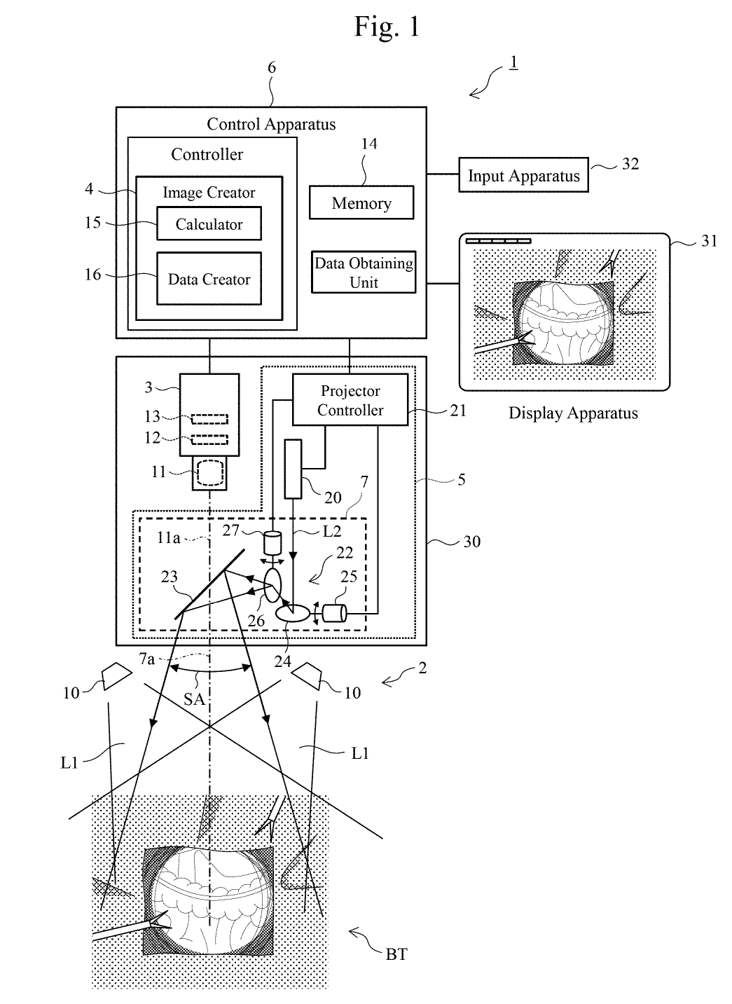

FIG. 1 is a drawing illustrating a configuration of an image processing system (also can be referred to as a medical assistance system or a projection system) according to this embodiment. An image processing system 1 irradiates a tissue (an irradiated body) BT (for example, an organ: FIG. 1 illustrates an organ as an example of the tissue BT) of an organism (for example, an animal) with infrared light (hereinafter, when referred to as “infrared light” in this description, the concept encompasses “near-infrared light”), detects light radiated from the tissue BT, and displays information (for example, an image) on the tissue BT using the detection result on a screen of a display apparatus. For example, the image processing system 1 has a function to directly project the image regarding the tissue BT on the tissue BT. The light radiated from the tissue BT of the organism includes, for example, light (for example, infrared light) obtained by irradiating the tissue BT with the infrared light (for example, light in a wavelength range, for example, from 800 nm to 2400 nm) and fluorescent emitted by irradiating the tissue BT labelled with a luminous substance such as fluorescent dye with excitation light. The information (for example, the image) on the tissue BT to be projected may be present on the tissue BT by two or more pieces of information.

The image processing system 1 is applicable to, for example, an abdominal operation in a surgical operation. For example, the image processing system 1 according to the embodiment is an image processing system for surgery assistant or a medical image processing system. The image processing system 1 displays an image photographed through the irradiation of the infrared light on the display apparatus or displays the photographed image obtained through the irradiation of the light (for example, visible light and infrared light) and information on an affected part analyzed using the infrared light on the screen of the display apparatus, and projects this information on the affected part directly or indirectly on the affected part in the tissue BT. The image processing system 1 can display an image illustrating components of the tissue BT as the image regarding the tissue BT. The image processing system 1 can display an image highlighting a region including a specific component in the tissue BT as the image regarding the tissue BT. Such image is, for example, an image illustrating a distribution of lipid and a distribution of water content in the tissue BT. The image processing system 1 can also overlap the image regarding the affected part with at least a part of the affected part and display the image. An operator can perform a surgery or a similar operation while directly seeing the information displayed on the affected part by the image processing system 1. The operator can also perform the surgery while seeing the photographed image of the tissue BT displayed on the screen of the display apparatus or a composite image produced by combining the information on the affected part. An operating person (a person in operation: can also be simply referred to as a “user”) of the image processing system 1 may be a person identical to the operator (the person in operation) or may be another person (such as a person in charge of support and a person engaged in medical treatment).

As one example, the image processing system 1 includes an irradiator (an infrared light irradiating apparatus) 2, an optical detector 3, a projector (a projecting apparatus and an information irradiator) 5, a control apparatus 6, a display apparatus 31, and an input apparatus 32. The control apparatus (the image processing apparatus) 6 is constituted of a CPU (a processor) and a computer, includes a controller including an image creator 4 and a memory (storage apparatus) 14, and controls their behaviors in the image processing system 1. The following describes an overview of their behaviors and configurations.

(i) Irradiator 2

The irradiator 2 irradiates the tissue BT of the organism with a detection light L1. The irradiator 2 includes a light source 10 that emits infrared light as one example. The light source 10 includes, for example, an infrared LED (an infrared-emitting diode) and emits infrared light as the detection light L 1. Compared with a laser light source, the light source 10 emits the infrared light with a wide wavelength range. As one example, the light source 10 emits the infrared light in the wavelength range including a first wavelength, a second wavelength, and a third wavelength. For example, the control apparatus 6 controls the emission of the infrared light in each wavelength range. The first wavelength, the second wavelength, and the third wavelength, which will be described later, are wavelengths used to calculate information on a specific component in the tissue BT. The light source 10 may include a solid light source other than the LED and may include a lamp light source such as a halogen lamp.

For example, the light source 10 is fixed such that a region irradiated with the detection light (an irradiated region with the detection light) does not move. The tissue BT is arranged at the irradiated region with the detection light. For example, the light source 10 and the tissue BT are arranged such that the relative position is not changed. In this embodiment, the light source 10 is supported independent from the optical detector 3 and supported independent from the projector 5. The light source 10 may be fixed integrally with at least one of the optical detector 3 and the projector 5.

(ii) Optical Detector 3

The optical detector 3 detects light (radiated light and detection light) radiated from the tissue BT irradiated with the detection light L1. The light via the tissue BT includes at least a part of light reflected by the tissue BT, light transmitting the tissue BT, and light scattered on the tissue BT. In this embodiment, as one example, the optical detector 3 is an infrared sensor that detects the infrared light reflected by and scattered on the tissue BT. The optical detector 3 may also be a sensor that detects light other than the infrared light.

In this embodiment, as one example, the optical detector 3 separately detects the infrared light at the first wavelength, the infrared light at the second wavelength, and the infrared light at the third wavelength. As one example, the optical detector 3 includes a photographing optical system (a detecting optical system) 11, an infrared filter 12, and an image sensor 13.

As one example, the photographing optical system 11 includes one or two or more optical elements (for example, lens) and can form the image (the photographed image) of the tissue BT irradiated with the detection light L1. As one example, the infrared filter 12 lets the infrared light in a predetermined wavelength range among the lights passing through the photographing optical system 11 through and cuts off the infrared light in a wavelength range other than the predetermined wavelength range. As one example, the image sensor 13 detects at least a part of the infrared light radiated from the tissue BT via the photographing optical system 11 and the infrared filter 12.

The image sensor 13, like a CMOS sensor or a CCD sensor, for example, includes a plurality of two-dimensionally arrayed light receiving elements. These light receiving elements are sometimes referred to as pixels or sub-pixels. As one example, the image sensor 13 includes a photodiode, a reading circuit, and an A/D converter. The photodiode is a photoelectric conversion element disposed at each light receiving element and generates an electric charge by the infrared light entered into the light receiving element. The reading circuit reads the electric charge accumulated in the photodiode from each light receiving element and outputs an analog signal indicative of an electric charge amount. The A/D converter converts the analog signals read by the reading circuit into digital signals. The image sensor 13 may be one that includes light receiving elements detecting only the infrared light or may be one that includes light receiving elements configured to detect the visible light in addition to the infrared light. In the former case, the display apparatus 31 displays an infrared image as the photographed image of the entire tissue BT. In the latter case, the display apparatus 31 displays any one of (may be selectable by the operating person) a visible image and the infrared image as the photographed image of the entire tissue BT.

As one example, the infrared filter 12 includes a first filter, a second filter, and a third filter. The first filter, the second filter, and the third filter mutually differ in the wavelength of the transmitting infrared light. The first filter lets the infrared light at the first wavelength through but cuts off the infrared lights at the second wavelength and the third wavelength. The second filter lets the infrared light at the second wavelength through but cuts off the infrared lights at the first wavelength and the third wavelength. The third filter lets the infrared light at the third wavelength through but cuts off the infrared lights at the first wavelength and the second wavelength.

The first filter, the second filter, and the third filter are arranged according to the array of the light receiving elements such that the infrared lights entering the respective light receiving elements pass through any one of the first filter, the second filter, and the third filter. For example, the infrared light at the first wavelength passing through the first filter enters a first light receiving element in the image sensor 13. The infrared light at the second wavelength passing through the second filter enters a second light receiving element adjacent to the first light receiving element. The infrared light at the third wavelength passing through the third filter enters a third light receiving element adjacent to the second light receiving element. Thus, the image sensor 13 detects optical intensities of the infrared light at the first wavelength, the infrared light at the second wavelength, and the infrared light at the third wavelength radiated from one part on the tissue BT by the adjacent three light receiving elements.

In this embodiment, the optical detector 3 outputs the detection results by the image sensor 13 as the digital signals (hereinafter referred to as photographed image data) in an image format. In the following description, the image photographed by the image sensor 13 is appropriately referred to as a photographed image. The data of the photographed image is referred to as the photographed image data. Here, while the photographed image is assumed to be in a Full High Definition format (an HD format) for convenience of explanation, the number of pixels and a pixel array (an aspect ratio) of the photographed image, a tone of a pixel value, and a similar specification are not limited.

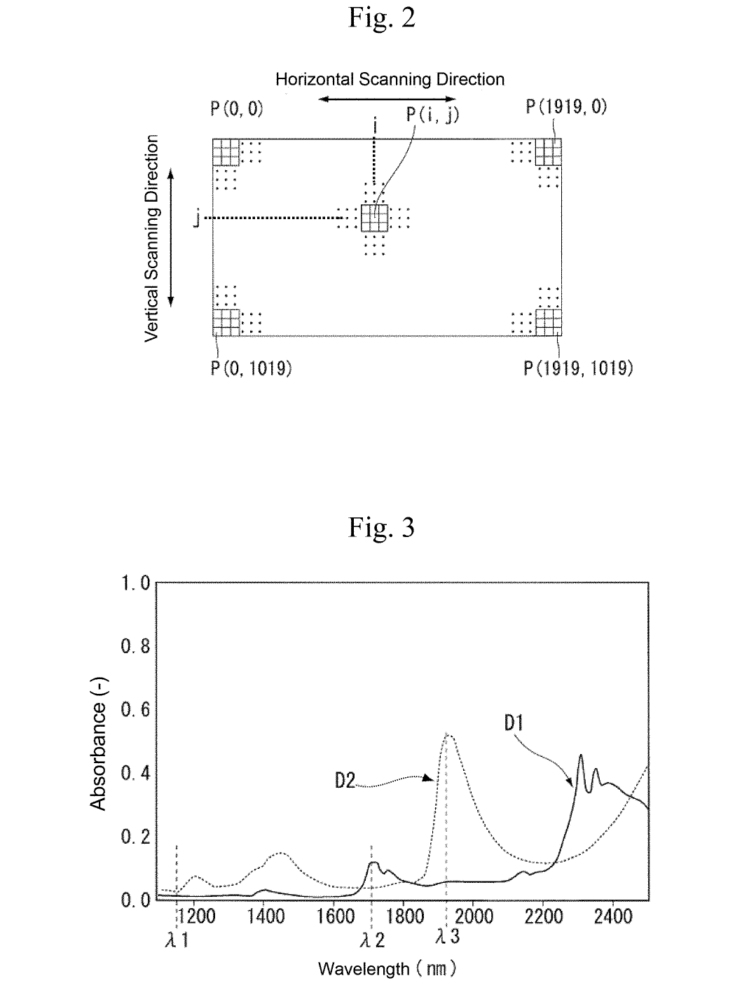

FIG. 2 is a conceptual diagram illustrating an example of the pixel array of the image. In the image in the HD format, 1920 pixels are aligned in a horizontal scanning direction and 1080 pixels are aligned in a vertical scanning direction. The plurality of pixels aligned in one row in the horizontal scanning direction are sometimes referred to as a horizontal scanning line. The pixel value of each pixel is, for example, represented by eight-bit data and is represented by 256 tones from 0 to 255 in decimal.

As described above, since the wavelength of the infrared light detected by each light receiving element in the image sensor 13 is determined by the position of the light receiving element, each pixel value of the photographed image data is associated with the wavelength of the infrared light detected by the image sensor 13. Here, the position of the pixel on the photographed image data is expressed by (i, j) and the pixel arranged at (i, j) is expressed by P(i, j). The i is the number of the pixel where the pixel at one end in the horizontal scanning direction is defined as 0 and the number goes in the ascending order like 1, 2, and 3 as approaching the other end. The j is the number of the pixel where the pixel at one end in the vertical scanning direction is defined as 0 and the number goes in the ascending order like 1, 2, and 3 as approaching the other end. The image in the HD format employs positive integers from 0 to 1919 for i and positive integers from 0 to 1079 for j.

A first pixel corresponding to the light receiving element in the image sensor 13 detecting the infrared light at the first wavelength is, for example, a pixel group meeting i=3N for a positive integer N. A second pixel corresponding to the light receiving element detecting the infrared light at the second wavelength is, for example, a pixel group meeting i=3N+1. A third pixel corresponding to the light receiving element detecting the infrared light at the third wavelength is a pixel group meeting i=3N+2.

(iii) Control Apparatus 6

The control apparatus 6, for example, sets a condition for a photographing process by the optical detector 3. The control apparatus 6, for example, controls an aperture ratio of a diaphragm disposed at the photographing optical system 11. The control apparatus 6, for example, controls a timing at which exposure to the image sensor 13 starts and a timing at which the exposure ends. Thus, the control apparatus 6 controls the optical detector 3 to cause the optical detector 3 to photograph the tissue BT irradiated with the detection light L1. The control apparatus 6, for example, includes a data obtaining unit (may also referred to as a data receiving unit) that obtains the photographed image data showing the photographing result by the optical detector 3 from the optical detector 3. The control apparatus 6 includes the memory 14 and causes the memory 14 to store the photographed image data. The memory 14 stores various kinds of information such as data (projection image data) created by the image creator 4 and data indicative of settings of the image processing system 1 in addition to the photographed image data.

The image creator 4 disposed in the control apparatus 6 creates image data regarding the tissue BT using the detection result by the optical detector 3 obtained by the data obtaining unit. The image data regarding the tissue BT includes, for example, the photographed image data of the entire tissue BT and data of a component image (for example, an image of a site where an amount of water content is large) corresponding to the affected part. The projection image projected on the tissue BT, as will be described later, is created by the image creator 4 in the controller performing an arithmetic operation on the detection result by the optical detector 3.

As one example, the image creator 4 includes a calculator 15 and a data creator 16. The calculator 15 uses a distribution of the optical intensity for the wavelength of the light (such as the infrared light and the fluorescent) detected by the optical detector 3 to calculate the information on the component of the tissue BT. Here, the following describes a method of calculating the information on the component of the tissue BT. FIG. 3 is a graph illustrating a distribution D1 of absorbance of a first substance and a distribution D2 of absorbance of a second substance in a near-infrared wavelength region. For example, the first substance is lipid and the second substance is water in FIG. 3. The graph in FIG. 3 indicates the absorbance by the vertical axis and the wavelength [nm] by the horizontal axis.

Any wavelength is settable to a first wavelength λ1. For example, the first wavelength λ1 is set to a wavelength at which the absorbance is relatively small in the distribution of the absorbance of the first substance (the lipid) in the near-infrared wavelength region and the absorbance is relatively small in the distribution of the absorbance of the second substance (the water) in the near-infrared wavelength region. Energy of the infrared light at the first wavelength λ1 absorbed into the lipid is low, and the optical intensity radiated from the lipid is high. Additionally, energy of the infrared light at the first wavelength λ1 absorbed into the water is low, and the optical intensity radiated from the water is high.

Any wavelength different from the first wavelength λ1 is settable to a second wavelength λ2. The second wavelength λ2, for example, is set to a wavelength at which the absorbance of the first substance (the lipid) is higher than the absorbance of the second substance (the water). When an object (for example, a tissue) is irradiated with the infrared light at the second wavelength λ2, the energy absorbed into the object becomes large and the optical intensity radiated from this object becomes weak as a ratio of the lipid to the water contained in this object increases. For example, when the ratio of the lipid contained in a first part of the tissue is larger than that of the water, the energy of the infrared light at the second wavelength λ2 absorbed into the first part of the tissue becomes large and the optical intensity radiated from this first part becomes weak. For example, when the ratio of the lipid contained in a second part of the tissue is smaller than that of the water, the energy of the infrared light at the second wavelength λ2 absorbed into the second part of the tissue becomes low and the optical intensity radiated from this second part becomes intense compared with the first part.

Any wavelength different from both of the first wavelength λ1 and the second wavelength λ2 is settable to a third wavelength λ3. The third wavelength λ3, for example, is set to a wavelength at which the absorbance of the second substance (the water) is higher than the absorbance of the first substance (the lipid). When the object is irradiated with the infrared light at the third wavelength λ3, the energy absorbed into the object becomes large and the optical intensity radiated from this object becomes weak as a ratio of the water to the lipid contained in this object increases. For example, in contrast to the above-described case of the second wavelength λ2, when the ratio of the lipid contained in the first part of the tissue is larger than that of the water, the energy of the infrared light at the third wavelength λ3 absorbed into the first part of the tissue becomes low, and the optical intensity radiated from this first part becomes high. For example, when the ratio of the lipid contained in the second part of the tissue is smaller than that of the water, the energy of the infrared light at the third wavelength λ3 absorbed into the second part of the tissue becomes large and the optical intensity radiated from this second part becomes weak compared with the first part.

The calculator 15 uses the photographed image data output from the optical detector 3 to calculate the information on the component of the tissue BT. In this embodiment, the wavelength of the infrared light detected by each light receiving element in the image sensor 13 is determined by the positional relationships between the respective light receiving elements and the infrared filter 12 (from the first to the third filters). The calculator 15 uses a pixel value P1 corresponding to the output from the light receiving element detecting the infrared light at the first wavelength, a pixel value P2 corresponding to the output from the light receiving element detecting the infrared light at the second wavelength, and a pixel value P3 corresponding to the output from the light receiving element detecting the infrared light at the third wavelength among the photographing pixels (see FIG. 2) to calculate the distribution of the lipid and the distribution of the water content contained in the tissue BT.

Here, the pixel P(i, j) in FIG. 2 is assumed as the pixel (the pixel value P1) that corresponds to the light receiving element detecting the infrared light at the first wavelength λ1 in the image sensor 13. The pixel P(i+1, j) is assumed as the pixel (the pixel value P2) that corresponds to the light receiving element detecting the infrared light at the second wavelength λ2. The pixel P(i+2, j) is assumed as the pixel (the pixel value P3) that corresponds to the light receiving element detecting the infrared light at the third wavelength λ3 in the image sensor 13.

The calculator 15 uses these pixel values to calculate an index Q(i, j).

For example, the calculated index Q is an index indicative of a ratio of the amount of lipid to the amount of water at the part photographed at the pixel P(i, j), the pixel P(i+1, j), and the pixel P(i+2, j) in the tissue BT. For example, the large index Q(i, j) suggests the large amount of lipid and the small index Q(i, j) suggests the large amount of water.

The calculator 15 thus calculates the index Q(i, j) at the pixel P(i, j). While changing the values of i and j, the calculator 15 calculates indexes at other pixels to calculate the distribution of the index. For example, since a pixel P(i+3, j), similar to the pixel P(i, j), corresponds to the light receiving element detecting the infrared light at the first wavelength in the image sensor 13, the calculator 15 uses the pixel value of the pixel P(i+3, j) instead of the pixel value of the pixel P(i, j) to calculate the indexes at the other pixels. For example, the calculator 15 uses the pixel value of the pixel P(i+3, j) equivalent to the detection result of the infrared light at the first wavelength, the pixel value of a pixel P(i+4, j) equivalent to the detection result of the infrared light at the second wavelength, and a pixel value of a pixel P(i+5, j) equivalent to the detection result of the infrared light at the third wavelength to calculate an index Q(i+1, j).

The calculator 15 calculates the indexes Q(i, j) of the respective pixels regarding the plurality of pixels to calculate the distribution of the index. The calculator 15 may calculate the indexes Q(i, j) regarding all pixels in a range in which the pixel values required to calculate the indexes Q(i, j) are included in the photographed image data. The calculator 15 may calculate the indexes Q(i, j) regarding a part of the pixels and calculate the distribution of the index Q(i, j) by interpolation operation using the calculated indexes Q(i, j).

The index Q(i, j) calculated by the calculator 15 does not become a positive integer in general. Therefore, the data creator 16 in FIG. 1 appropriately rounds values to convert the index Q(i, j) into data in a predetermined image format. For example, the data creator 16 uses the result calculated by the calculator 15 to create the image data regarding the component of the tissue BT. In the following description, the image regarding the component of the tissue BT is appropriately referred to as the component image (or the projection image). The data of the component image is referred to as component image data (or projection image data).

Here, while the component image is assumed to be the component image in the HD format as illustrated in FIG. 2 for convenience of explanation, the number of pixels and a pixel array (an aspect ratio) of the component image, a tone of a pixel value, and a similar specification are not limited. The component image may be in an image format identical to that of the photographed image and may be in an image format different from that of the photographed image. When creating the data of the component image in the image format different from that of the photographed image, the data creator 16 appropriately performs the interpolation process.

The data creator 16 calculates a value found by converting the index Q(i, j) into, for example, the eight-bit (256 tones) digital data as the pixel value of the pixel P(i, j) in the component image. For example, the data creator 16 divides the index Q(i, j) by transmission constant, which uses an index equivalent to one gradation of the pixel value, and rounds the division value off to the closest whole number to convert the index Q(i, j) into the pixel value of the pixel P(i, j). In this case, the pixel values are calculated so as to meet an approximately linear relationship with the indexes.

As described above, the calculation of the index regarding the one pixel using the pixel values of the three pixels in the photographed image possibly results in insufficient pixels required to calculate the index regarding the pixels at an end of the photographed image. Consequently, the indexes required to calculate the pixel value of the pixel at the end of the component image becomes insufficient. Thus, in the case where the indexes required to calculate the pixel values of the pixels in the component image become insufficient, the data creator 16 may calculate the pixel values of the pixels in the component image by interpolation or a similar method. In such case, the data creator 16 may set the pixel values of the pixels in the component image that cannot be calculated due to the insufficient indexes to a predetermined value (for example, 0).

The method of converting the index Q(i, j) into the pixel value can be appropriately changed. For example, the data creator 16 may calculate the component image data such that the pixel values and the indexes have a nonlinear relationship. The data creator 16 may set a value found by converting the index calculated using the pixel value of the pixel P(i, j), the pixel value of the pixel P(i+1, j), and the pixel value of the pixel P(i+2, j) in the photographed image into the pixel value as the pixel value of the pixel P(i+1, j).

The data creator 16 may set the pixel value for the index Q(i, j) to a constant value when the value of the index Q(i, j) is less than a lower limit value of a predetermined range. This constant value may also be the minimum tone (for example, 0) of the pixel value. The pixel value for the index Q(i, j) may also be set to a constant value when the value of the index Q(i, j) exceeds an upper limit value of the predetermined range. This constant value may be the maximum tone (for example, 255) of the pixel values or may be the minimum tone (for example, 0) of the pixel values.

Since the calculated index Q(i, j) becomes large as the amount of lipid at the region increases, the pixel value of the pixel P(i, j) increases as the amount of lipid at the region increases. For example, since the large pixel value generally corresponds to the bright display of the pixel, as the amount of lipid at the region increases, the region is brightly highlighted for display.

Meanwhile, persons in operation possibly demand the bright display on the region where the amount of water is large. Therefore, as one example, the image processing system 1 has a first mode that brightly highlights and displays the information on the amount of first substance (lipid) and a second mode that brightly highlights and displays the information on the amount of second substance (water). The memory 14 stores setting information indicative of whether any modes of the first mode and the second mode is set to the image processing system 1.

With the first mode set as the mode, the data creator 16 creates first component image data found by converting the index Q(i, j) into the pixel value of the pixel P(i, j). The data creator 16 creates second component image data found by converting an inverse of the index Q(i, j) into the pixel value of the pixel P(i, j). As the amount of water increases in the tissue, the value of the index Q(i, j) becomes small, and the value of the inverse of the index Q(i, j) becomes large. Therefore, the pixel value (the tone) of the pixel corresponding to the region where the amount of water is large increases in the second component image data.

As one example, with the second mode set as the mode, the data creator 16 may calculate a difference value found by subtracting the pixel value converted from the index Q(i, j) from a predetermined tone as the pixel value of the pixel P(i, j). For example, with a pixel value converted from the index Q(i, j) of 50, the data creator 16 may calculate 205, which is found by subtracting 50 from the maximum tone (for example, 255) of the pixel value as the pixel value of the pixel P(i, j).

The image creator 4 stores the created component image data in the memory 14. The control apparatus 6 supplies the component image data created by the image creator 4 to the projector 5 and causes the projector 5 to project the component image on the tissue BT to highlight the specific part (for example, the above-described first part and second part) in the tissue BT. The control apparatus 6 controls a timing at which the projector 5 projects the component image. The control apparatus 6 controls the brightness of the component image projected by the projector 5. The control apparatus 6 can cause the projector 5 to stop projecting the image. The control apparatus 6 can control the start and stop of the projection of the component image such that the component image is displayed on the tissue BT in flash to highlight the specific part in the tissue BT.

### (iv) Projector 5

The projector 5 includes a projection optical system 7 that scans the tissue BT with a visible light L2 based on this data and projects the image (the projection image) on the tissue BT by scanning with the visible light L2. The projector 5 may be configured as an apparatus independent as the projecting apparatus.

The projector 5 is, for example, a scanning projection system to scan light (for example, first light and projection light) on the tissue BT and, as one example, includes a light source 20, the projection optical system (an irradiation optical system) 7, and a projector controller 21. For example, the light source 20 emits the visible light at a predetermined wavelength different from the detection light L1. The light source 20 includes a laser diode and emits laser beam as the visible light. The light source 20 emits the laser beam at optical intensity according to a current supplied from the outside. For example, the projector 5 may project the information on the tissue BT such as the image and the diagram on the tissue BT through the irradiation of the first light (for example, the visible light and the infrared light) and may project information on another tissue BT (for example, the image and the diagram) on the tissue BT with second light (for example, the visible light and the infrared light) different from the first light while irradiating the first light. For example, the projector 5 may project the information on the tissue BT on the tissue BT through the irradiation of the first light (for example, the visible light and the infrared light) at the first wavelength and may project information on another tissue BT (for example, the image and the diagram) on the tissue BT with the second light at the second wavelength different from the first light while irradiating the first light.

The projection optical system 7 guides the laser beam emitted from the light source 20 onto the tissue BT and scans the tissue BT with this laser beam. As one example, the projection optical system 7 includes a scanner 22 and a wavelength selection mirror 23. The scanner 22 can deflect the laser beam emitted from the light source 20 in two directions. For example, the scanner 22 is an optical system of a reflection system. The scanner 22 includes a first scanning mirror 24, a first driver 25 that drives the first scanning mirror 24, a second scanning mirror 26, and a second driver 27 that drives the second scanning mirror 26. For example, the respective first scanning mirror 24 and second scanning mirror 26 are a galvanometer mirror, an MEMS mirror, or a polygon mirror.

The first scanning mirror 24 and the first driver 25 are, for example, horizontal scanners that deflect the laser beam emitted from the light source 20 in the horizontal scanning direction. The first scanning mirror 24 is arranged at a position where the laser beam emitted from the light source 20 enters. The first driver 25 is controlled by the projector controller 21 to turn the first scanning mirror 24 based on a drive signal received from the projector controller 21. The laser beam emitted from the light source 20 is reflected by the first scanning mirror 24 and deflects in a direction according to an angular position of the first scanning mirror 24. The first scanning mirror 24 is arranged on an optical path of the laser beam emitted from the light source 20.

The second scanning mirror 26 and the second driver 27 are, for example, vertical scanners that deflect the laser beam emitted from the light source 20 in the vertical scanning direction. The second scanning mirror 26 is arranged at a position where the laser beam reflected by the first scanning mirror 24 enters. The second driver 27 is controlled by the projector controller 21 to turn the second scanning mirror 26 based on a drive signal received from the projector controller 21. The laser beam reflected by the first scanning mirror 24 is reflected by the second scanning mirror 26 and deflects in a direction according to an angular position of the second scanning mirror 26. The second scanning mirror 26 is arranged on the optical path of the laser beam emitted from the light source 20.

The horizontal scanner and vertical scanner are each, for example, a galvanometer scanner. The vertical scanner may have a configuration similar to that of the horizontal scanner or a configuration different from that of the horizontal scanner. For example, a scanning method using the scanners according to the embodiment may be a raster scan method that scans the entire screen region using horizontal scanning and vertical scanning in combination or may be a vector scan method that performs only a required line drawing. The raster scan method performs the scanning in the horizontal direction at a frequency higher than that of the scanning in the vertical direction. Therefore, the galvanometer mirror may be used for the scanning in the vertical scanning direction and the MEMS mirror or the polygon mirror, which behaves at a frequency higher than that of the galvanometer mirror, may be used for the scanning in the horizontal scanning direction.

The wavelength selection mirror (a wavelength selector) 23 is an optical member that guides the laser beam deflected by the scanner 22 onto the tissue BT. The laser beam reflected by the second scanning mirror 26 is reflected by the wavelength selection mirror 23 and irradiated on the tissue BT. This embodiment arranges the wavelength selection mirror 23, for example, on the optical path between the tissue BT and the optical detector 3. The wavelength selection mirror 23 is, for example, a dichroic mirror or a dichroic prism. For example, the wavelength selection mirror 23 has a property where the detection light emitted from the light source 10 in the irradiator 2 transmits and the visible light emitted from the light source 20 in the projector 5 is reflected. For example, the wavelength selection mirror 23 has a property where light in an infrared region transmits and light in a visible region is reflected.

This embodiment assumes that an optical axis 7a of the projection optical system 7 is an axis (an identical optical axis) coaxial with the laser beam passing through a center of a scanning range SA in which the projection optical system 7 performs the scanning with the laser beam. As one example, the optical axis 7a of the projection optical system 7 is coaxial with the laser beam passing through the center in the horizontal scanning direction by the first scanning mirror 24 and the first driver 25 and passing through the center in the vertical scanning direction by the second scanning mirror 26 and the second driver 27. For example, the optical axis 7a on the light-emitting side of the projection optical system 7 is coaxial with the laser beam passing through the center of the scanning range SA on the optical path between an optical member arranged on the side closest to an irradiation target (for example, the tissue BT) with the laser beam in the projection optical system 7 and the irradiation target. In this embodiment, at least the optical axis 7a on the light-emitting side of the projection optical system 7 among the optical axes of the projection optical system 7 is coaxial with the laser beam passing through the center of the scanning range SA on the optical path between the wavelength selection mirror 23 and the tissue BT.

In this embodiment, an optical axis 11a in the photographing optical system 11 is, for example, coaxial with a rotational center axis of a lens included in the photographing optical system 11. This embodiment configures that at least some of the optical axes of the photographing optical system 11 are mutually coaxial with at least some of the optical axes of the projection optical system 7 on a predetermined optical path (or at least some of the optical paths). For example, the optical axis 11a of the photographing optical system 11 is configured coaxially with the optical axis 7a on the light-emitting side of the projection optical system 7. For example, the optical axis of the light (for example, the light radiated from the tissue BT) passing through the optical path of the photographing optical system 11 is configured coaxially with the optical axis of the light (for example, the first light) passing through the optical path of the projection optical system 7 on a common optical path through which at least the mutual lights (the light radiated from the tissue BT and the light irradiated to the tissue BT) pass. Accordingly, even if the user changes a photographing position of the tissue BT, the use of the image processing system 1 according to the embodiment can project the component image to be projected on the tissue BT without positional displacement. In this embodiment, a chassis 30 houses the respective optical detector 3 and projector 5. The respective optical detector 3 and projector 5 are fixed to the chassis 30. Therefore, the positional displacement between the optical detector 3 and the projector 5 is reduced, thereby reducing the positional displacement between the optical axis 11a of the photographing optical system 11 and the optical axis 7a of the projection optical system 7.

The projector controller 21 controls the current supplied to the light source 20 according to the pixel values. For example, to display the pixel (i, j) in the component image, the projector controller 21 supplies the current according to the pixel value of the pixel (i, j) to the light source 20. As one example, the projector controller 21 performs amplitude modulation on the current supplied to the light source 20 according to the pixel values. The projector controller 21 controls the first driver 25 to control the position where the laser beam enters at each time in the horizontal scanning direction in the scanning range of the laser beam by the scanner 22. The projector controller 21 controls the second driver 27 to control the position where the laser beam enters at each time in the vertical scanning direction in the scanning range of the laser beam by the scanner 22. As one example, the projector controller 21 controls the optical intensity of the laser beam emitted from the light source 20 according to the pixel value of the pixel (i, j) and controls the first driver 25 and the second driver 27 such that the laser beam enters the position equivalent to the pixel (i, j) in the scanning range.

### (v) Display Apparatus 31

The display apparatus 31 is coupled to the control apparatus 6, and, for example, constituted of a flat panel display such as a liquid crystal display. The control apparatus 6 can cause the display apparatus 31 to display, for example, the photographed image and the setting of the behavior of the image processing system 1. The control apparatus 6 can display the photographed image photographed by the optical detector 3 or an image produced through image processing on the photographed image on the display apparatus 31. The control apparatus 6 can display the component image (for example, the image showing the site (the affected part) containing the much water content) created by the image creator 4 or the image produced through image processing on the component image on the display apparatus 31. Furthermore, the control apparatus 6 can display the composite image produced by performing a composition process on the component image and the photographed image on the display apparatus 31.

When at least one of the photographed image and the component image is displayed on the display apparatus 31, the timing may be identical to or different from the timing at which the projector 5 projects the component image. For example, the control apparatus 6 may cause the memory 14 to store the component image data and supply the component image data stored in the memory 14 to the display apparatus 31 when the input apparatus 32 receives an input signal indicative of the display on the display apparatus 31.

The control apparatus 6 may cause the display apparatus 31 to display the image of the tissue BT photographed by a photographing apparatus having sensitivity to the wavelength range of the visible light. Alternatively, the control apparatus 6 may cause the display apparatus 31 to display at least one of the component image and the photographed image together with such image.

### (vi) Input Apparatus 32

The input apparatus 32 is coupled to the control apparatus 6, and, for example, constituted of a changeover switch, a computer mouse, a key board, and a touch-panel pointing apparatus (operated with a stylus pen and a finger). The input apparatus 32 can input the setting information to set the behavior of the image processing system 1. The control apparatus 6 can detect that the input apparatus 32 is operated. For example, the control apparatus 6 can change the setting of the image processing system 1 and causes the respective apparatuses in the image processing system 1 to execute processes according to information (input information) input via the input apparatus 32.

For example, when the user performs an input to specify the first mode, which brightly displays the information on the amount of lipid by the projector 5, to the input apparatus 32, the control apparatus 6 controls the data creator 16 to cause the data creator 16 to create the component image data according to the first mode. When the user performs an input to specify the second mode, which brightly displays the information on the amount of water by the projector 5, to the input apparatus 32, the control apparatus 6 controls the data creator 16 to cause the data creator 16 to create the component image data according to the second mode. Thus, the image processing system 1 can switch the mode between the first mode and the second mode, which highlight and display the component image projected by the projector 5.

The control apparatus 6, for example, can control the projector controller 21 in accordance with the input signal via the input apparatus 32 and cause the projector 5 to start, cancel, or resume the display of the component image. The control apparatus 6, for example, can control the projector controller 21 in accordance with the input signal via the input apparatus 32 and adjust at least one of the color and the brightness of the displayed component image by the projector 5. For example, there may be a case where the tissue BT has coloring in which reddish is strong derived from, for example, a blood. In such case, displaying the component image by a color (for example, green) in a complementary color relationship with the tissue BT eases visual identification between the tissue BT and the component image.

<Function 1: Basic Image Display Functions>

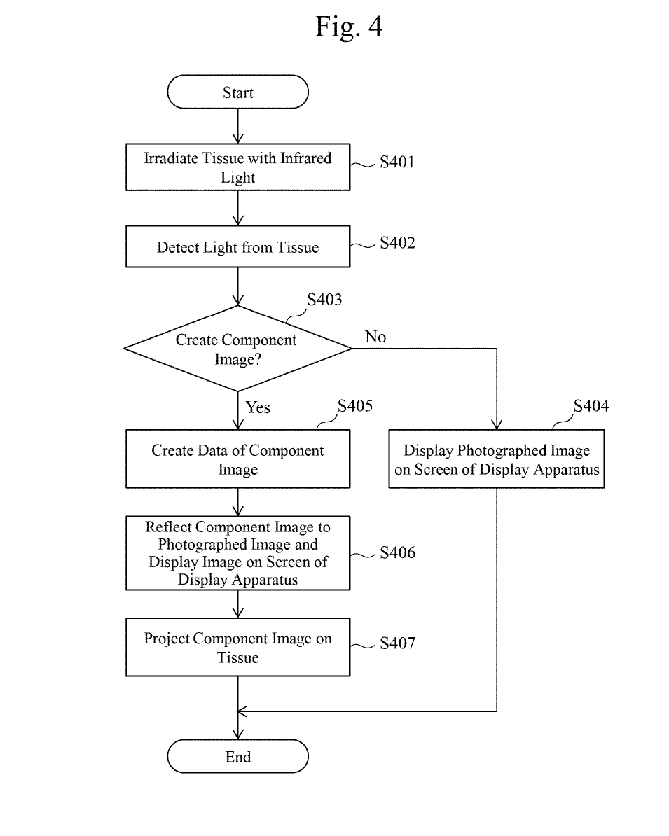

Function 1 according to the embodiment is the basic image display functions in the image processing system 1. Function 1 has functions of displaying the photographed image of the entire tissue BT on the screen of the display apparatus 31 or displaying the composite image obtained by combining the component image (the image showing the affected site containing the much water content) with the photographed image on the screen of the display apparatus 31 and projecting the component image on the tissue BT. FIG. 4 is a flowchart describing process contents in Function 1 according to this embodiment.

### (i) Step 401

In response to an instruction to start a photographing behavior of the tissue BT from the control apparatus 6, the irradiator 2 irradiates the tissue BT with the detection light (for example, the infrared light).

### (ii) Step 402

The optical detector 3 detects the light (for example, the infrared light) radiated from the tissue BT irradiated with the detection light. When the image sensor 13 in the optical detector 3 can detect the visible light as well, the optical detector 3 detects the infrared light and the visible light. The photographed image of the entire tissue BT is formed from the detected light, and the control apparatus 6 stores the data of this photographed image in the memory 14.

(iii) Step 403

The control apparatus 6 determines whether the component image needs to be created or not. Regarding the necessity to create the component image, for example, the operating person (the operator) inputs the necessity for creation to the input apparatus 32. When the control apparatus 6 determines that the component image needs not to be created (NO at Step 403), the process transitions to Step 404. When the control apparatus 6 determines that the component image needs to be created (YES at Step 403), the process transitions to Step 405.

### (iv) Step 404

The control apparatus 6 reads the photographed image of the tissue BT from the memory 14, transmits the photographed image to the display apparatus 31, and instructs the display apparatus 31 to display the photographed image on the screen. The display apparatus 31 displays the received photographed image on the screen in response to this instruction. The displayed photographed image may be the infrared image or may be the visible image.

### (v) Step 405

The control apparatus 6 uses the calculator 15 included in the image creator 4 to calculate component information (the information on the component of the tissue BT) on the amount of lipid and the amount of water content in the tissue BT. For example, as described above, the calculator 15 calculates the indexes Q(i, j) of the respective pixels regarding the plurality of pixels to calculate the distribution of the index. The calculator 15 converts the index Q(i, j) into the pixel value of the pixel P(i, j). Since the index Q(i, j) becomes large as the amount of lipid at the region increases, the pixel value of the pixel P(i, j) increases as the amount of lipid at the region increases. Meanwhile, the region where the amount of water content is large produces the outcome opposite to the case of lipid.

Therefore, as the component image, while the region where the amount of lipid is large is displayed brightly, the region where the amount of water content is large is displayed darkly in general. As described above, performing the predetermined operation makes it possible to display the region where the amount of water content is large brightly.

### (vi) Step 406

With the photographed image as the infrared image, the control apparatus 6 reflects the component image to the infrared image and transmits the image to the display apparatus 31, and the display apparatus 31 displays the received image on the screen. Meanwhile, with the photographed image as the visible image, the control apparatus 6 combines the component image with the visible image and transmits the composite image to the display apparatus 31, and the display apparatus 31 displays the received composite image on the screen.

(vii) Step 407

The control apparatus 6 transmits the component image data created at Step 405 to the projector 5 and instructs the projector 5 to project this component image on the tissue BT. The projector 5 scans the tissue BT based on this component image data with the visible light and projects the component image on the tissue BT. As described above, for example, the image processing system 1 two-dimensionally (two directions) and sequentially scans with the visible light using the two scanning mirrors (for example, the first scanning mirror 24 and the second scanning mirror 26) based on the component image data to ensure projecting the component image on the tissue BT.

<Operational Advantages and the like by Function 1>

The image processing system 1 according to this embodiment, for example, scans the tissue BT by the scanner 22 with the laser beam to directly project (draw) the image (for example, the component image) showing the information on the tissue BT on the tissue BT. The laser beam generally has high parallelism, and a change in spot size of the laser beam relative to a change in optical path length is small. Therefore, the image processing system 1 can project the clear image with little blur on the tissue BT regardless of unevenness on the tissue BT.

The image processing system 1 includes the optical axis 11a of the photographing optical system 11 and the optical axis 7a of the projection optical system 7 configured to be coaxial. Therefore, even when the relative position between the tissue BT and the optical detector 3 changes, the positional displacement between the part of the tissue BT photographed by the optical detector 3 and the part on which the image is projected by the projector 5 is lowered. For example, a parallax between the image projected by the projector 5 and the tissue BT is lowered.

The image processing system 1 projects the component image on which the specific region of the tissue BT is highlighted as the image showing the information on the tissue BT. Therefore, the person in operation can perform treatments such as an incision, a resection, and a drug administration on the specific region while seeing the component image projected on the tissue BT. Since the image processing system 1 can change the color and the brightness of the component image, the image processing system 1 can display the component image so as to be easily identified visually from the tissue BT. Like this embodiment, when the projector 5 directly irradiates the tissue BT with the laser beam, a flicker referred to as a speckle, which is visually perceived easily, is generated on the component image projected on the tissue BT. This speckle allows the user to easily identify the component image from the tissue BT.

The image processing system 1 may set a period to project one frame of the component image to be variable. For example, the projector 5 can project the images by 60 frames per second, and the image creator 4 may create the image data such that an all black image in which all pixels are displayed darkly is included between the component image and the next component image. In this case, the component image is likely to be visually perceived flickery and therefore is easily identified from the tissue BT.

The display of the component image on the screen of the display apparatus 31 and the projection of the component image on the tissue BT may be executed in real-time. For example, while the operating person (such as the operator and an examiner) conducts a medical practice on the tissue BT, the component image is displayed and projected in real-time. The operating person (such as the operator and the examiner) can confirm whether the target specific region has been treated.

<Function 2: Function to Project Input Diagram on Monitor on Tissue BT with Visible Light Laser>

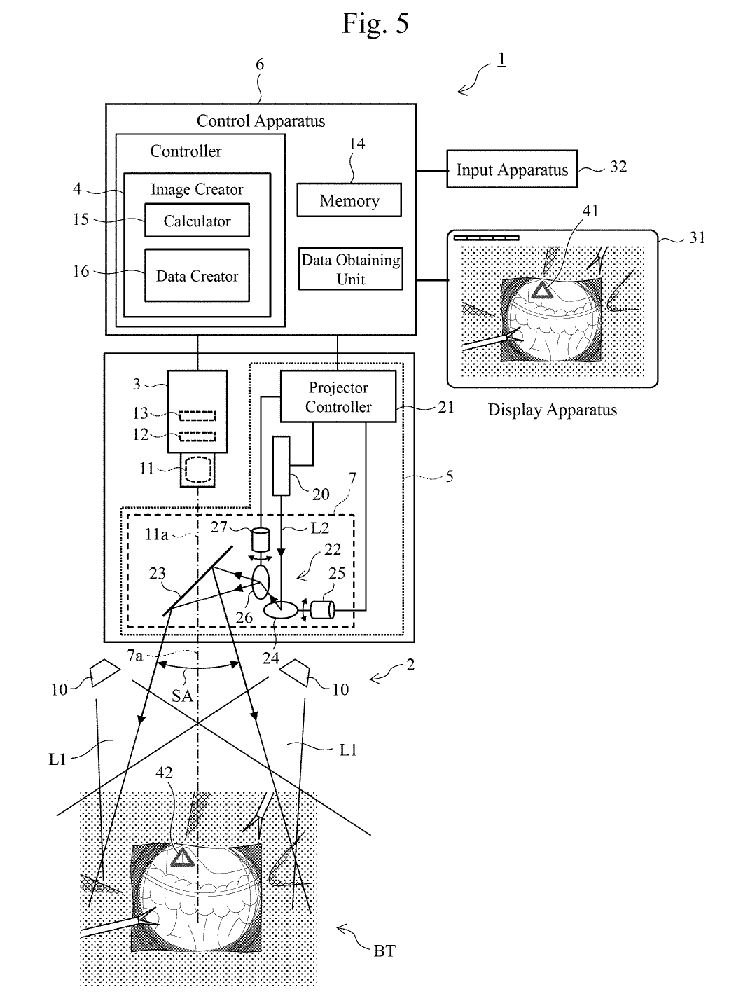

Function 2 according to the embodiment is one of special functions by the image processing system 1 and is a function that projects a diagram identical to a diagram input to the photographed image of the entire tissue BT displayed on the screen of the display apparatus 31 on the tissue BT. Additionally, the image processing system 1 includes a function of displaying the composite image obtained by combining the photographed image or the component image of the tissue BT with the photographed image on the screen of the display apparatus 31 and a function of projecting (irradiating) marking information (contents of inputs such as diagrams, lines, and characters) input to the input apparatus 32 on the tissue BT with light. FIG. 5 is a drawing describing the overview of this Function 2. The configuration of the image processing system 1 illustrated in FIG. 5 is identical to that in FIG. 1 and therefore the following omits the detailed explanation of the configuration.

The image processing system 1 according to this embodiment first displays the photographed image of the entire tissue BT on the screen of the display apparatus 31. In this respect, the component image may be reflected to the photographed image and displayed on the screen or only the photographed image may be displayed on the screen. The photographed image may be the infrared image or may be the visible image. The component image may be projected on the tissue BT, or the projection may be omitted.

With the photographed image (such as a still image and a moving image) of the tissue BT displayed on the screen of the display apparatus 31, when the operating person (for example, the operator) inputs (draws) a marking (such as any diagram) 41 to a site (for example, the affected part and the highlighted part) related to the surgery and examination using the input apparatus 32, the control apparatus 6 senses this input, starts controlling the projector 5 in response to this input, and projects a diagram 42 identical to the marking 41 on a position on the tissue BT identical to the position to which the marking 41 has been input with the visible light. For example, while the photographed image (such as the still image and the moving image) of the tissue BT is displayed on the screen of the display apparatus 31, when the operating person inputs the marking 41 to the photographed image using the input apparatus 32, the control apparatus 6 senses this input and controls a behavior (a projection behavior) of the light irradiation by the projector 5 based on the input. Then, the control apparatus 6 causes the projector 5 to project the marking (for example, the diagram 42) identical to the marking 41 input to the photographed image to the position on the tissue BT corresponding to the position to which the marking 41 has been input with the light to irradiate the marking with a light. Thus projecting the diagram identical to the diagram input to the display screen on the approximately identical position on the tissue BT allows the operating person to appropriately and easily treat (such as the surgery and the examination) the site on the tissue BT corresponding to the site confirmed on the screen. As one example, the control apparatus 6 may control the projector 5 such that the projector 5 projects (irradiates) a first marking (such as any line and diagram) input by the operating person on the tissue BT with the first light (for example, the visible light and the infrared light) and further projects (irradiates) a second marking (such as any line and diagram) input by the operating person on the tissue BT with the second light (for example, the visible light and the infrared light). The number of irradiated lights is not limited to two (two kinds) and may be equal to or more than three (three kinds).

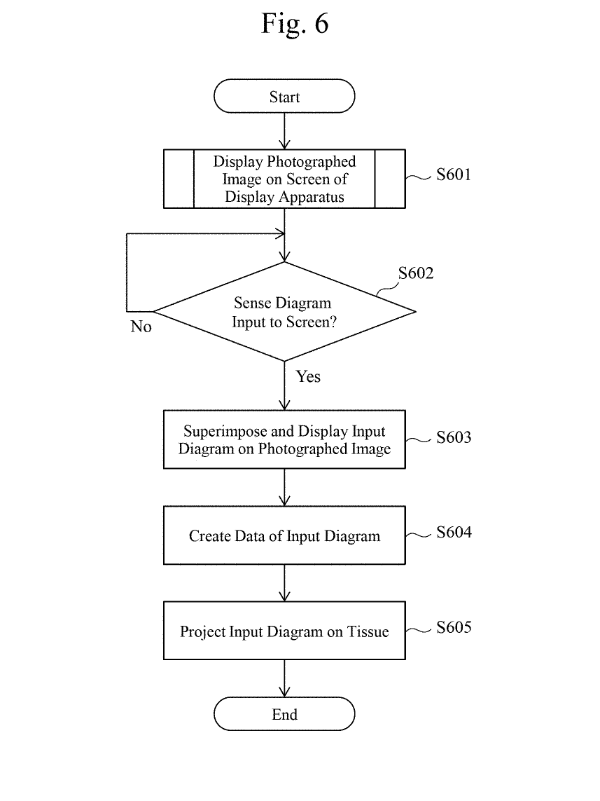

FIG. 6 is a flowchart describing process contents in Function 2 according to this embodiment.

### (i) Step 601

The process at Step 601 is identical to the process shown in the flowchart in FIG. 4, which displays the photographed image on the screen of the display apparatus 31, reflects the component image to the photographed image, and displays the image on the screen. In this case, the component image may be projected on the tissue BT or the component image may be displayed only on the screen. The following omits the detailed explanation of Step 601.

### (ii) Step 602

A processor (not illustrated) in the display apparatus 31 stands by until the input of the diagram (the marking 41) to the displayed photographed image by the operating person is sensed. When the processor senses this input of the diagram, the process transitions to Step 603. The processor in the display apparatus 31 retains information (coordinate information) of the position of the input diagram (the marking 41) and the pixel values of the input diagram (the marking 41) in a memory (not illustrated). The pixel values (the brightness) and the color of the diagram (the marking 41) can be preset. For example, the operating person may input (a touch input) the diagram using the stylus pen, the finger, or a similar tool or may input the diagram using audio.

Here, while the one example where the processor in the display apparatus 31 senses the input of the diagram has been explained, the control apparatus 6 may sense the input to the screen in the display apparatus 31.

(iii) Step 603

The display apparatus 31 superimposes and displays the input diagram (the marking 41) on the photographed image.

### (iv) Step 604

The control apparatus 6 receives (as described above, the control apparatus 6 directly senses the signal generated by the diagram input by the operating person in some cases) a signal (an input signal) generated by the diagram input to the display apparatus 31 by the operating person from the display apparatus 31, obtains the position information and the information on the pixel values of the input diagram (the marking 41), and creates diagram data to be projected on the tissue BT. Note that the pixel values of the diagram (a mark 42) projected on the tissue BT need not to be identical to the diagram (the marking 41) displayed on the screen of the display apparatus 31 and may be appropriately adjustable by the operating person (the operator). This is because, for example, in the operating room, the tissue BT is considerably brightly illuminated by a surgery shadowless lamp, and therefore the pixel values identical to those of the diagram (the marking 41) displayed on the screen possibly make it difficult to see the diagram data even projected on the tissue BT.

### (v) Step 605

The control apparatus 6 transmits the input diagram data created at Step 604 to the projector 5 and instructs the projector 5 to project this diagram on the tissue BT. The projector 5 receives the instruction of diagram projection and the input image data to be projected from the control apparatus 6, scans the tissue BT with the visible light based on the information on the irradiation position (the projection position) of the visible light on the tissue BT identified using the position information in the photographed image of this input diagram data and the pixel values in this photographed image, and projects the diagram 42 on the tissue BT. The projection behavior is as described above and therefore the detailed explanation is omitted here.

<Function 3: Function to Automatically Surround Affected Site by Outline (Affected Site Profile Display Function)>

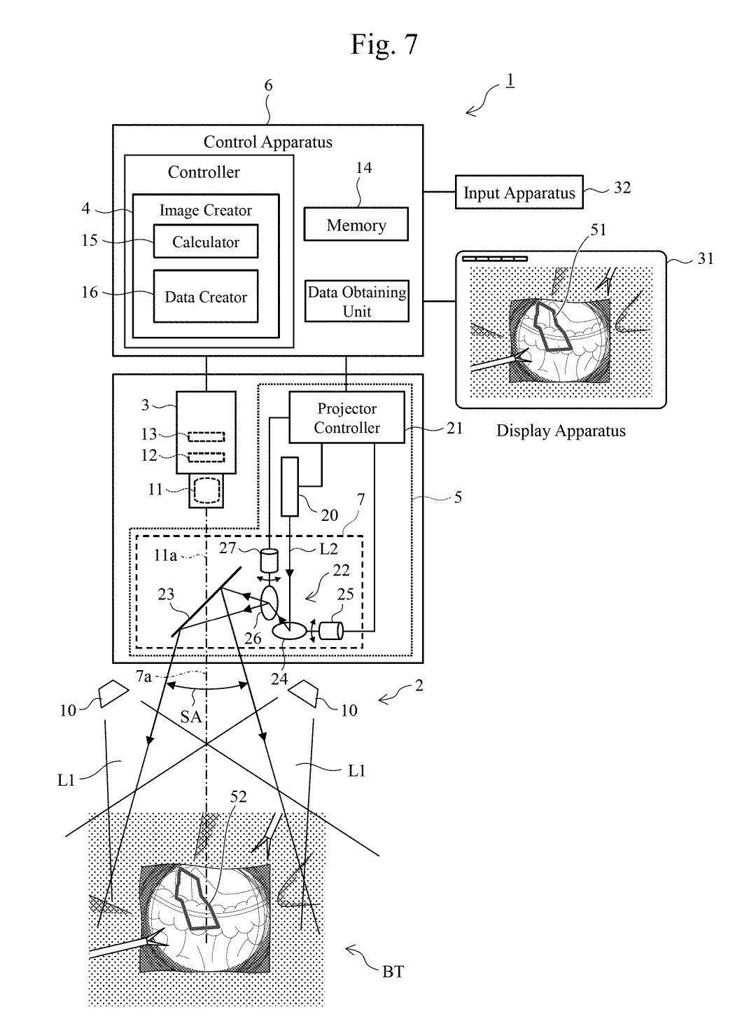

Function 3 is one of the special functions by the image processing system 1 and is a function that automatically surrounds and displays the affected site (for example, all or a part of the affected part) in the photographed image of the entire tissue BT displayed on the screen of the display apparatus 31 by outline and automatically projects this outline showing the affected site also on the tissue BT. FIG. 7 is a drawing describing the overview of this Function 3. The configuration of the image processing system 1 illustrated in FIG. 7 is identical to that in FIG. 1 and therefore the following omits the detailed explanation of the configuration.

The image processing system 1 according to this embodiment displays the photographed image of the entire tissue BT on the screen of the display apparatus 31. In this respect, the component image may be reflected to the photographed image and displayed on the screen or only the photographed image may be displayed on the screen. The photographed image may be the infrared image or may be the visible image. The component image may be projected on the tissue BT, or the projection may be omitted.

In this image processing system 1, the data creator 16 in the control apparatus 6 obtains position information of the profile of the component image based on the component image data and creates outline data. Since the outline is displayed with a predetermined width, the position information of the pixels included in this predetermined width is obtained. The created outline data is transmitted to the display apparatus 31, and the outline surrounding the affected site is displayed on the screen.

The control apparatus 6 transmits the created outline data to the projector 5. The projector 5 draws (projects) the outline on the tissue BT with the visible light such that the outline surrounds the affected site based on the received outline data. Thus, the affected site is surrounded by the outline with a predetermined thickness and automatically displayed on the screen and this outline is automatically projected on the tissue BT. This ensures further highlighting the affected site; therefore, the operating person (such as the operator and the examiner) can further appropriately and easily recognize the part to be operated and the part to be examined.

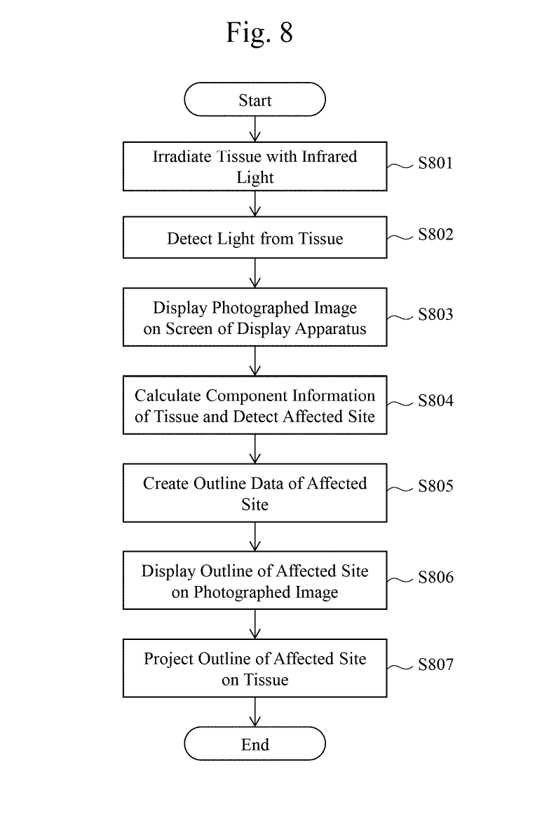

FIG. 8 is a flowchart describing process contents in Function 3 according to this embodiment.

### (i) Step 801

In response to an instruction to start the photographing behavior of the tissue BT from the control apparatus 6, the irradiator 2 irradiates the tissue BT with the detection light (the infrared light).

### (ii) Step 802

The optical detector 3 detects the light (the infrared light) radiated from the tissue BT irradiated with the detection light. When the image sensor 13 in the optical detector 3 can detect the visible light as well, the optical detector 3 detects the infrared light and the visible light. The photographed image of the entire tissue BT is formed from the detected light, and the control apparatus 6 stores this photographed image in the memory 14.

(iii) Step 803

As one example, the control apparatus 6 reads the photographed image of the tissue BT from the memory 14, transmits the photographed image to the display apparatus 31, and instructs the display apparatus 31 to display the photographed image on the screen. The display apparatus 31 displays the received photographed image on the screen in response to this instruction. The displayed photographed image may be the infrared image or may be the visible image.

### (iv) Step 804

For example, the control apparatus 6 uses the calculator 15 included in the image creator 4 to calculate the component information (information on the component of the tissue BT) on the amount of lipid and the amount of water content in the tissue BT. As described above, the calculator 15 calculates the indexes Q(i, j) of the respective pixels regarding the plurality of pixels to calculate the distribution of the index. The calculator 15 converts the index Q(i, j) into the pixel value of the pixel P(i, j). Since the index Q(i, j) becomes large as the amount of lipid at the region increases, the pixel value of the pixel P(i, j) increases as the amount of lipid at the region increases. The region where the amount of water content is large produces the outcome opposite to the case of lipid.

As described above, the part of the large amount of water content is detected, and this part is detected as the affected site. The data of the affected site corresponds to the above-described component image data.

### (v) Step 805

Using the data creator 16, the control apparatus 6 obtains the position information of the profile of the affected site based on affected site data and creates the outline data. Since the outline is displayed with a predetermined width, the position information of the pixels included in this predetermined width is obtained. The outline data is transmitted to the display apparatus 31 and the projector 5.

### (vi) Step 806

The display apparatus 31 performs superimposition display of the outline data of the affected site on the photographed image.

(vii) Step 807

The projector 5 projects the outline of the affected site on the tissue BT based on the received outline data with the visible light. The projection behavior is as described above and therefore the detailed explanation is omitted here.

<Function 4: Function to Surround and Display Plurality of Site Candidates for Affected Part by Outline on Screen and Project Selected Sites on Tissue BT>

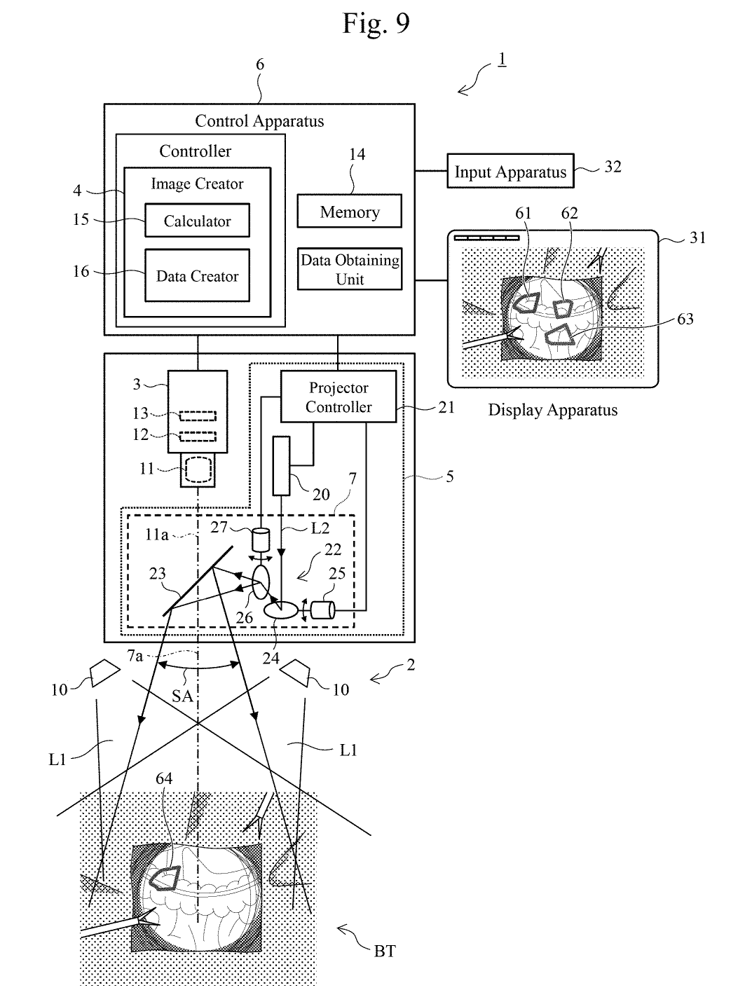

Function 4 is one of the special functions by the image processing system 1 and is a function that automatically surrounds and displays the plurality of respective candidates for affected part by outline in the photographed image of the entire tissue BT displayed on the screen of the display apparatus 31, deletes outlines other than a selected candidate for affected part in response to the selection (the input by the operating person) by the operating person, and automatically projects the outline of this selected candidate for affected part also on the tissue BT. FIG. 9 is a drawing describing the overview of this Function 4. The configuration of the image processing system 1 illustrated in FIG. 9 is identical to that in FIG. 1 and therefore the following omits the detailed explanation of the configuration.

The image processing system 1 according to this embodiment displays the photographed image of the entire tissue BT on the screen of the display apparatus 31. In this respect, the component image may be reflected to the photographed image and displayed on the screen or only the photographed image may be displayed on the screen. The photographed image may be the infrared image or may be the visible image. The component image may be projected on the tissue BT, or the projection may be omitted.

As one example, in the image processing system 1, the calculator 15 in the control apparatus 6 calculates the component information and identifies a site containing the amount of water content by equal to or more than the predetermined amount. This site containing the amount of water content by equal to or more than the predetermined amount is detected as the site candidate for affected part. It is assumed that the plurality of site candidates for affected part are detected here.

The data creator 16 in the control apparatus 6 obtains the component image data of the sites containing the amount of water content equal to or more than the predetermined amount from the calculator 15. For example, the data creator 16 obtains the position information of the profiles of the plurality of site candidates for affected part based on this component image data and creates outline data. Since the outline is displayed with a predetermined width, the position information of the pixels included in this predetermined width is obtained. The created outline data is transmitted to the display apparatus 31, and the outlines surrounding the plurality of site candidates for affected part are displayed on the screen.

In response to the operating person (such as the operator and the examiner: can also be simply referred to as the “user”) selecting at least any one of the plurality of site candidates for affected part, the display apparatus 31 deletes outlines other than the outline of the selected site candidate for affected part. The information on the selected outline is transmitted to the control apparatus 6.

Meanwhile, the control apparatus 6 transmits the outline data corresponding to the selected outline to the projector 5. The projector 5 draws (projects) the outline on the tissue BT with the visible light such that the outline surrounds the affected site based on the received outline data. Thus, the plurality of site candidates for affected part are displayed on the photographed image in the screen, only the selected outline based on the selection by the operating person is left, the outlines other than the selected outline are deleted, and the selected outline is automatically projected also on the tissue BT. This ensures further highlighting the affected site desired by the operating person and allows the operating person to further appropriately and easily identify the part to be operated and the part to be examined. Accordingly, the image processing system 1 can assist the smooth progress of the surgery and the examination.

Note that Function 4 does not project the outlines of the site candidates for affected part on the tissue BT at the beginning. When a selection is made among the plurality of site candidates for affected part displayed on the screen, the outline of this selected site candidate for affected part is projected on the tissue BT. Meanwhile, in addition to the display of the outlines of the plurality of detected site candidates for affected part on the screen of the display apparatus 31, these outlines of the plurality of site candidates for affected part may be projected on the tissue BT as well. When the operating person selects the site candidate for affected part on the screen, the outlines other than the outline of the selected site may be deleted from the screen and the tissue BT.

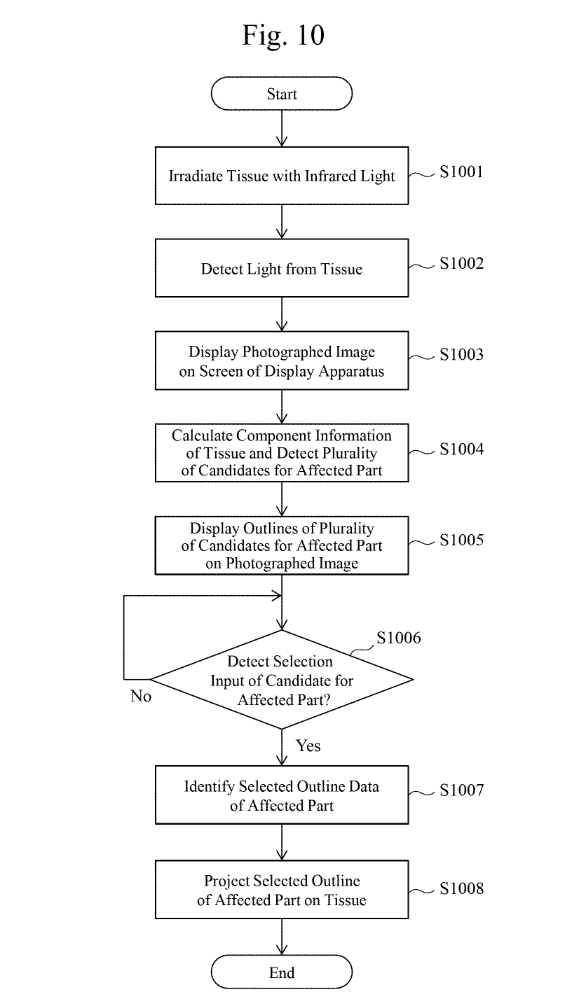

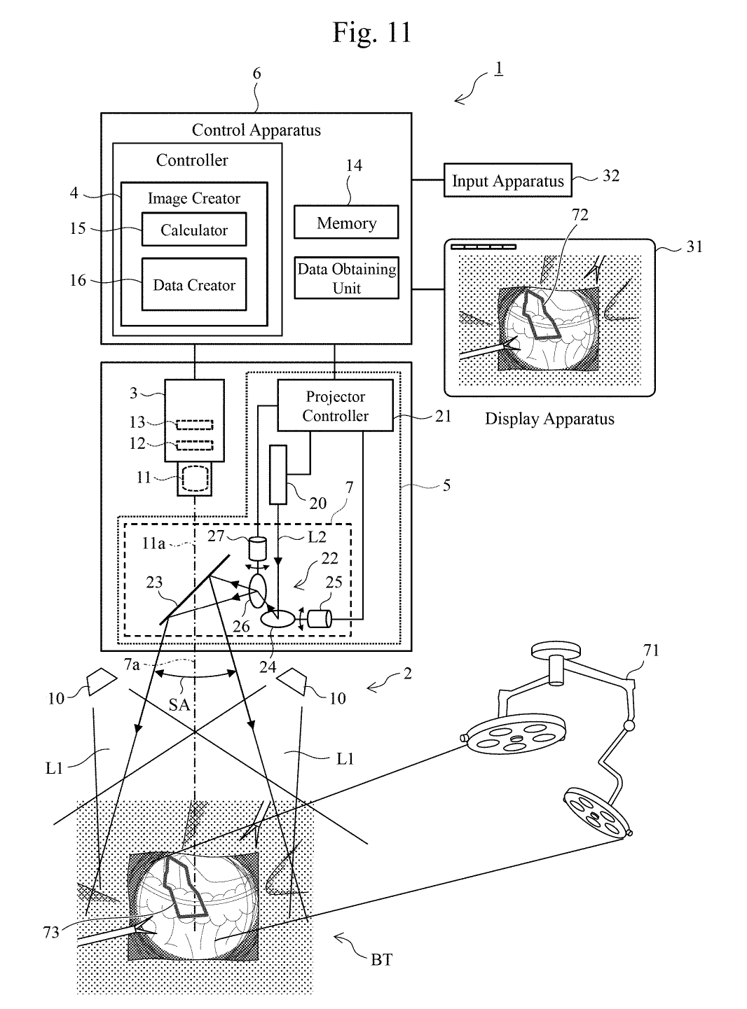

FIG. 10 is a flowchart describing process contents in Function 4 according to this embodiment.

### (i) Step 1001

In response to the instruction to start the photographing behavior of the tissue BT from the control apparatus 6, the irradiator 2 irradiates the tissue BT with the detection light (the infrared light).

### (ii) Step 1002

The optical detector 3 detects the light (the infrared light) radiated from the tissue BT irradiated with the detection light. When the image sensor 13 in the optical detector 3 can detect the visible light as well, the optical detector 3 detects the infrared light and the visible light. The photographed image of the entire tissue BT is formed from the detected light, and the control apparatus 6 stores this photographed image in the memory 14.

(iii) Step 1003

The control apparatus 6 reads the photographed image of the tissue BT from the memory 14, transmits the photographed image to the display apparatus 31, and instructs the display apparatus 31 to display the photographed image on the screen. The display apparatus 31 displays the received photographed image on the screen in response to this instruction. The displayed photographed image may be the infrared image or may be the visible image.

### (iv) Step 1004

The control apparatus 6 uses the calculator 15 included in the image creator 4 to calculate the component information (information on the component of the tissue BT) on the amount of lipid and the amount of water content in the tissue BT. As described above, the calculator 15 calculates the indexes Q(i, j) of the respective pixels regarding the plurality of pixels to calculate the distribution of the index. The calculator 15 converts the index Q(i, j) into the pixel value of the pixel P(i, j). Since the index Q(i, j) becomes large as the amount of lipid at the region increases, the pixel value of the pixel P(i, j) increases as the amount of lipid at the region increases. The region where the amount of water content is large produces the outcome opposite to the case of lipid.

As described above, as one example, the parts where the amount of water content and the amount of lipid are equal to or more than the predetermined values are detected, and these parts are detected as the site candidates for affected part.

### (v) Step 1005

Using the data creator 16, the control apparatus 6 obtains position information of the respective profiles of the site candidates for affected part based on the plurality of site candidates for affected part data and creates the respective outline data of the site candidates for affected part. Since the outline is displayed with a predetermined width, the position information of the pixels included in this predetermined width is obtained. For example, the respective outline data are stored in the memory 14 and transmitted to the display apparatus 31.

The display apparatus 31 performs the superimposition display of the outlines of the respective site candidates for affected part in the displayed photographed image based on the respective outline data. The colors and the display forms (a dotted line and a thickness) of the respective outlines may be changed and displayed for easy identification of the regions surrounded by the respective outlines. These colors and display forms may be configured to be settable in advance and changeable by the operating person.

### (vi) Step 1006

The processor (not illustrated) in the display apparatus 31 stands by until it senses the selection input of at least any one of the plurality of outlines displayed on the photographed image(the selection input is made by the operating person). When the processor senses this selection input, the process transitions to Step 1007. The processor in the display apparatus 31 retains information to identify the selected outline (identifier information of the selected outline when an identifier (ID) is given to the outline and position information (coordinate information) of this selected outline when the ID is not given) in the memory (not illustrated) and transmits the information to the control apparatus 6. The pixel values (the brightness) and the colors of the diagram (the marking 41) can be preset. For example, to select the outline displayed on the photographed image, the operating person may select (the touch input) the outline using the stylus pen, the finger, or a similar tool or may select the outline using audio.

Here, while the explanation that the processor in the display apparatus 31 senses the selection input by the operating person has been given, the control apparatus 6 may sense the input to the screen in the display apparatus 31.

(vii) Step 1007

The control apparatus 6 receives (as described above, the control apparatus 6 directly senses the signal generated by the selection input by the operating person in some cases) a signal (an input signal) generated by the selection of the outline displayed on the display apparatus 31 by the operating person from the display apparatus 31, identifies the corresponding outline data based on the information to identify the selected outline included in the input signal, and reads the outline data from the memory 14. This corresponding outline data is transmitted to the projector 5.

(viii) Step 1008

The projector 5 obtains the instruction of the projection of the outline data on the tissue BT and the outline data from the control apparatus 6 and projects the outline of the affected site on the tissue BT with the visible light based on the position information of the pixels forming this outline included in this outline data. The projection behavior is as described above and therefore the detailed explanation is omitted here.

<Functions 5 to 7: Functions to Highlight Outline (Guide Light) Projected (Drawn) on Tissue BT (Guide Light Highlight Function)>

The surgery shadowless lamp is constituted of a plurality of LED light sources and considerably bright, for example, maximum 160000 lux. In view of this, to project the outline (the guide light) on the tissue BT by Functions 2 to 4, it is sometimes difficult to determine the guide light by eyes of a human. To accentuate the guide light, increasing intensity and increasing energy density of the guide light are necessary. However, it is concerned that the irradiation of the intense guide light affects a human body; therefore, the irradiation of the guide light with low intensity as much as possible is preferable. Therefore, Functions 5 to 7 are provided for highlight display of the guide light (the outline) with the guide light (the visible light laser with a low output as much as possible) with the low intensity as much as possible.

### (i) Function 5

Function 5 is a function where the light source (a visible light laser light source) 20 irradiates the tissue BT while flashing a light source with a single color (a single wavelength) to project the outline and the light source 20 irradiates the tissue BT using the light sources with a plurality of colors (a plurality of wavelengths) while switching these light sources with the plurality of colors to project the outline.

FIG. 11 is a drawing describing Function 5 and illustrates a schematic configuration of the image processing system 1 according to this embodiment used in an operating room. The configuration of this image processing system 1 is identical to that in FIG. 1 and therefore the following omits the detailed explanation of the configuration.

As illustrated in FIG. 11, for example, Function 5 is used under an environment where a surgery shadowless lamp 71 is lit up in the operating room. Function 5 is a function used when Functions 2 to 4 project the diagram (the mark (the input diagram) 42 in FIG. 5 and outlines 52 and 64 in FIG. 7 and FIG. 9) on the tissue BT or when Functions 2 to 4 are about to perform the projection.

As described above, Function 5 has, for example, a flash mode, which flashes single-color visible light laser beam to highlight the guide light (an outline 73), and a color switching mode to switch the visible light laser beams with a plurality of colors to highlight the guide light (the outline 73). The modes used to highlight the guide light (the outline 73) are selectable with the input apparatus 32.

By the selection of a guide light highlight mode by Function 5, the control apparatus 6 instructs the projector 5 to highlight the guide light (the outline 73) in any of the modes and project the guide light on the tissue BT. The projector 5 performs the projection while flashing the guide light (the outline 73) that has been already projected on the tissue BT or the guide light (the outline 73) that will be projected on the tissue BT, or the projector 5 performs the projection while switching the colors.

In the case where the projector 5 projects the guide light on the tissue BT while switching the guide lights with the plurality of colors, the switching of the guide light (the outline 73) needs to be sensable for the human. That is, for example, even if the color is switched at every 1/30 seconds, the timing is identical to a unit of switching of frames; therefore, the switching of the color is blended into the switching of the frames and cannot be distinguished by the eyes of the human. Accordingly, this embodiment switches the colors, for example, in units of 0.1 to 2 seconds, preferably in units of 0.5 seconds.

### (ii) Function 6

Function 6 is a function, with the operator (the user) wearing shutter glasses 81 in the operating room with the surgery shadowless lamp 71 lit up, that synchronizes an opening/closing timing of shutters (for example, liquid crystal shutters) of these shutter glasses 81 with a lighting timing of the surgery shadowless lamp 71 and an irradiation timing of the guide light (the visible light laser) for highlight display of the guide light. One example of the shutter glasses 81, a wearable apparatus according to the embodiment, is liquid crystal shutter glasses. While the explanation is given with the shutter glasses here as one example, the shutter glasses 81 may be glasses that restrict an eyesight (a visual filed, a field of view) by switching (switching of light shielding/transmitting) between optical transmission and non-transmission in addition to the shutters. In that meaning, in addition to the glasses, the shutter glasses 81 need only to be a apparatus (an eyesight restricting apparatus: for example, a head mounted eyesight restricting apparatus) worn by the operator that restricts an eyesight of a left eye and an eyesight of a right eye of the operator in alternation.

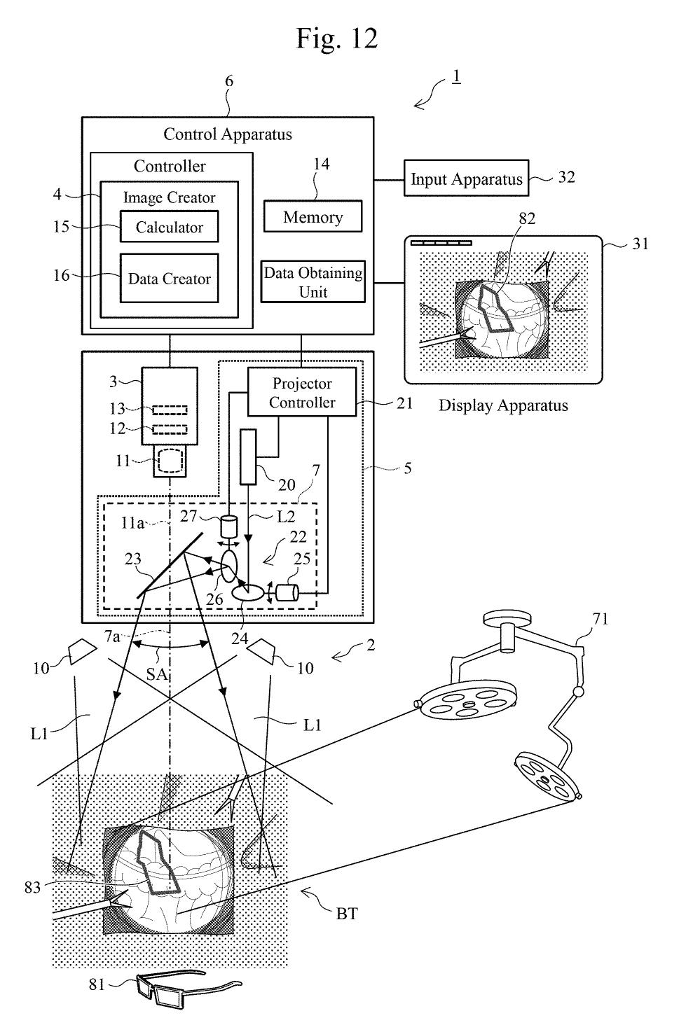

FIG. 12 illustrates a schematic configuration of the image processing system 1 according to this embodiment used in the operating room.

As illustrated in FIG. 12, for example, Function 6 is used under the environment where the surgery shadowless lamp 71 is lit up in the operating room similar to Function 5. Function 6 is a function used when Functions 2 to 4 project the diagram (the mark (the input diagram) 42 in FIG. 5 and the outlines 52 and 64 in FIG. 7 and FIG. 9) on the tissue BT or when Functions 2 to 4 are about to perform the projection.

To use the guide light highlight mode by Function 6, the operator wears the liquid crystal shutter glasses 81 and inputs an instruction to execute Function 6 from the input apparatus 32. The liquid crystal shutter glasses 81 and the control apparatus 6 are mutually communicable over, for example, wireless communications. Therefore, turning ON an open/close control of the liquid crystal shutters of the liquid crystal shutter glasses 81 allows the control apparatus 6 to obtain information on the opening/closing timing of the liquid crystal shutters. The control apparatus 6 controls the lighting timing of the surgery shadowless lamp 71 and the timing of the guide light irradiation based on the obtained information on the opening/closing timing of the liquid crystal shutters.

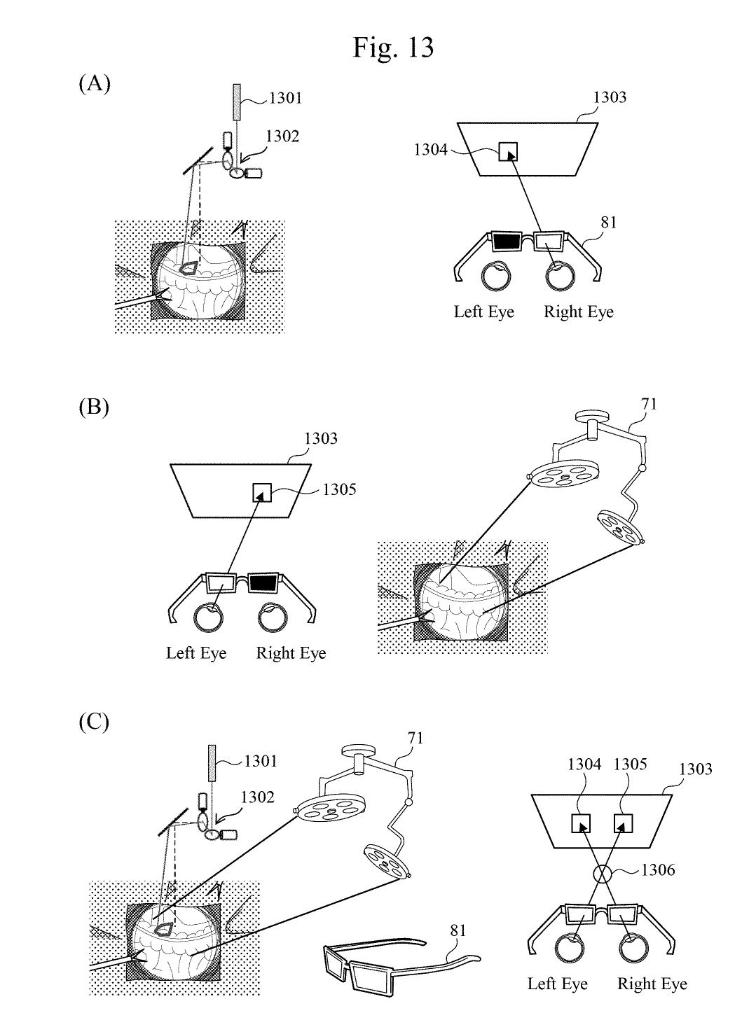

FIG. 13 is a drawing describing the overview of Function 6. FIG. 13(A) is a drawing illustrating a right eye video (an odd number field) 1304 displayed on a screen 1303 when the tissue BT is irradiated with guide light (a laser light source) 1301 for 1/60 seconds. FIG. 13(B) is a drawing illustrating a left eye video (an even number field) 1305 displayed on the screen 1303 when the surgery shadowless lamp 71 lights up the tissue BT for 1/60 seconds. FIG. 13(C) is a drawing illustrating an image 1306 to be played.

As illustrated in FIG. 13, the liquid crystal shutters are configured to be switched between the openings and closings of the left eye shutter and the right eye shutter in alternation, for example, in units of 1/60 seconds (see FIGS. 13(A) and 13(B)). As one example, with the right eye shutter opened, control is performed such that the tissue BT is irradiated with the guide light (the visible light laser) via the galvanometer mirror and the surgery shadowless lamp (the LED light source) 71 is lit out (turned OFF). For example, an image captured by the right eye constitutes the image 1304 with the odd number field and an image captured by the left eye constitutes the image 1305 with the even number field. Then, the images of the right eye and the left eye are seen to be combined in a pseudo manner, and thus the guide light 1301 is seen to be highlighted (see FIG. 13(C)). The combined image becomes the frame image (the played image) 1306 in units of 1/30.

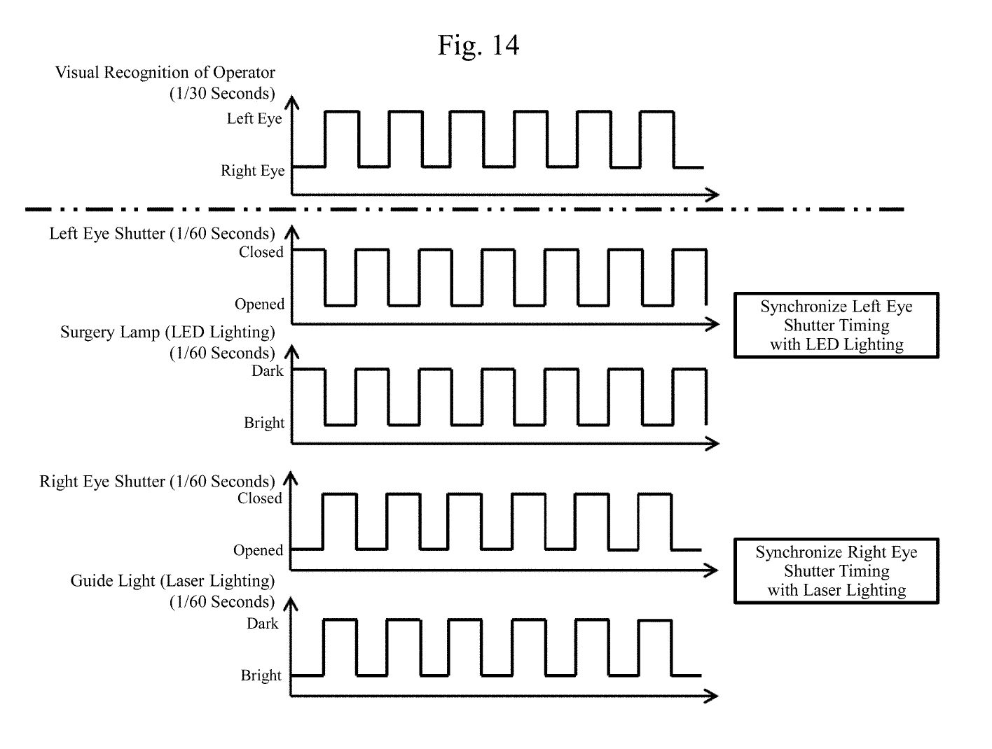

FIG. 14 is a timing chart illustrating switching timings to open/close the liquid crystal shutters of the liquid crystal shutter glasses 81, a lighting timing of the surgery shadowless lamp 71, and a timing of the guide light irradiation (projection). The control apparatus 6 communicates with the liquid crystal shutter glasses 81 and controls the opening/closing of the liquid crystal shutters in accordance with this timing chart. As illustrated in FIG. 14, the left eye shutter repeats the opening/closing at every 1/60 seconds. While the right eye shutter repeats the opening/closing at every 1/60 seconds as well, the opening/closing timing is opposite to that of the left eye shutter. While the left eye shutter is open, the surgery shadowless lamp 71 is turned ON (bright), and while the left eye shutter is close, the surgery shadowless lamp 71 is turned OFF (dark). While the right eye shutter is open, the guide light (the visible light laser) is turned ON (bright), and while the right eye shutter is close, the guide light (the visible light laser) is turned OFF (dark). That is, the control is performed such that the opening/closing timing of the left eye shutter becomes identical to the ON/OFF timing of the surgery shadowless lamp 71 and the opening/closing timing of the right eye shutter becomes identical to the ON/OFF timing of the guide light (the visible light laser). By thus performing the control, the images of the right eye and the left eye are seen to be combined in a pseudo manner. Consequently, the guide light is seen to be highlighted (the guide light is seen clearly and distinguishably).

The control apparatus 6 may perform the control so as to match the opening/closing timing of the left eye shutter with the ON/OFF timing of the guide light and match the opening/closing timing of the right eye shutter with the ON/OFF timing of the surgery shadowless lamp 71 through communications with the liquid crystal shutter glasses 81.

The roles of the left eye shutter and the right eye shutter may be switched at a predetermined timing. For example, the control apparatus 6 may perform the control so as to match the opening/closing timing of the left eye shutter with the ON/OFF timing of the guide light and match the opening/closing timing of the right eye shutter with the ON/OFF timing of the surgery shadowless lamp 71 for a certain period through communications with the liquid crystal shutter glasses 81 and the surgery shadowless lamp 71. The control apparatus 6 may perform the control so as to match the opening/closing timing of the left eye shutter with the ON/OFF timing of the surgery shadowless lamp 71 and match the opening/closing timing of the right eye shutter with the ON/OFF timing of the guide light, for example, after a lapse of the certain period. The control apparatus 6 repeats this control to switch the roles. This configuration avoids a problem of, for example, losing a sense of perspective to see the tissue BT by only one eye.

<Modifications>

(1) Time Division Control between Photographing Behavior and Drawing Behavior

Since the outline drawn (projected) on the tissue BT is displayed in the easily identified color, this outline possibly adversely affects the analysis of the image photographed by the optical detector 3.

Therefore, time-divisionally executing the photographing behavior by the optical detector 3 and the drawing behavior of the outline by the light source (the visible light laser) 20 ensures eliminating a possibility of the negative effect brought by the outline. The time division control is executed by, for example, repeating the photographing behavior and the drawing behavior in alternation at every 1/30 second.

As one example, the control apparatus 6 performs the control such that, for example, when the operating person inputs an instruction to start the behavior to the input apparatus 32, the control apparatus 6 senses this instruction to start the behavior, controls the optical detector 3 so as to execute the photographing behavior (a detection behavior) for first 1/30 seconds (during this period, the drawing behavior by the light source 20 in the projector 5 is stopped), and for the subsequent 1/30 seconds, stops the photographing behavior by the optical detector 3 such that the light source 20 in the projector 5 executes the drawing behavior of the outline. The control apparatus 6 repeats these behaviors to achieve the time division control between the photographing behavior and the drawing behavior. The ON/OFF of the time division control may be configured to be selectable by the operating person. The ON/OFF selection may be performed via the input apparatus 32.

### (2) Real-Time Display and Projection of Outline

As one example, the real-time display of the component image by Function 1 has been described above. The outline shows the profile of the affected site (including the candidates) corresponding to the component image; therefore, changing the component image also similarly changes the outline.

Accordingly, while any of Functions 3 to 7 is in use, displaying the outline in the screen and projecting the outline on the tissue BT in real-time changes the shape of the outline displayed and projected according to the resection or a similar operation on the affected site.

### (3) Modification of Irradiator 2

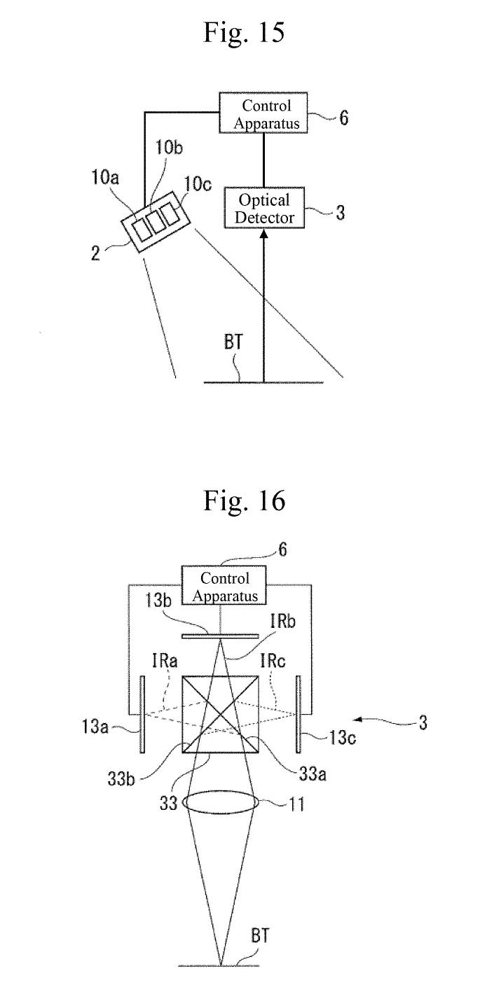

FIG. 15 is a drawing illustrating the modification of the irradiator 2. As one example, the irradiator 2 in FIG. 15 includes a plurality of light sources including a light source 10a, a light source 10b, and a light source 10c. As one example, all of the light source 10a, the light source 10b, and the light source 10c include LEDs emitting infrared light, and the wavelengths of the emitted infrared lights are mutually different. As one example, the light source 10a emits infrared light in a wavelength range containing the first wavelength but not containing the second wavelength and the third wavelength. As one example, the light source 10b emits infrared light in a wavelength range containing the second wavelength but not containing the first wavelength and the third wavelength. As one example, the light source 10c emits infrared light in a wavelength range containing the third wavelength but not containing the first wavelength and the second wavelength.

The control apparatus 6 can control the respective lightings and extinctions of the light source 10a, the light source 10b, and the light source 10c. For example, the control apparatus 6 sets the irradiator 2 in a first state in which the light source 10a is lit up and the light source 10b and the light source 10c are lit out. In the first state, the tissue BT is irradiated with the infrared light at the first wavelength emitted from the irradiator 2. While setting the irradiator 2 in the first state, the control apparatus 6 causes the optical detector 3 to photograph the tissue BT and obtains image data (photographed image data) in which the tissue BT irradiated with the infrared light at the first wavelength is photographed from the optical detector 3.

The control apparatus 6 sets the irradiator 2 in a second state in which the light source 10b is lit up and the light source 10a and the light source 10c are lit out. While setting the irradiator 2 in the second state, the control apparatus 6 causes the optical detector 3 to photograph the tissue BT and obtains photographed image data of the tissue BT irradiated with the infrared light at the second wavelength from the optical detector 3. The control apparatus 6 sets the irradiator 2 in a third state in which the light source 10c is lit up and the light source 10a and the light source 10b are lit out. While setting the irradiator 2 in the third state, the control apparatus 6 causes the optical detector 3 to photograph the tissue BT and obtains photographed image data of the tissue BT irradiated with the infrared light at the third wavelength from the optical detector 3.

The image processing system 1 can project the image (for example, the component image) showing the information on the tissue BT on the tissue BT with the configuration applying the irradiator 2 illustrated in FIG. 5 as well. The image processing system 1 photographs the tissue BT by the image sensor 13 (see FIG. 1) in each wavelength range, facilitating securing the resolution.

### (4) Modification of Optical Detector 3

While the optical detector 3 batch-detects the infrared light at the first wavelength, the infrared light at the second wavelength, and the infrared light at the third wavelength by the identical image sensor 13, the configuration is not limited to this. FIG. 16 is a drawing illustrating the modification of the optical detector 3. As one example, the optical detector 3 in FIG. 16 includes the photographing optical system 11, a wavelength separator 33, and a plurality of image sensors including an image sensor 13a, an image sensor 13b, and an image sensor 13c.

The wavelength separator 33 disperses the light radiated from the tissue BT by a difference in wavelength. The wavelength separator 33 in FIG. 16 is, for example, a dichroic prism. The wavelength separator 33 includes a first wavelength separation film 33a and a second wavelength separation film 33b. The first wavelength separation film 33a has a property where an infrared light IRa at the first wavelength is reflected and an infrared light IRb at the second wavelength and an infrared light IRc at the third wavelength transmit. The second wavelength separation film 33b is disposed so as to intersect with the first wavelength separation film 33a. The second wavelength separation film 33b has a property where the infrared light IRc at the third wavelength is reflected and the infrared light IRa at the first wavelength and the infrared light IRb at the second wavelength transmit.

Among the infrared lights IR radiated from the tissue BT, the infrared light IRa at the first wavelength is reflected by the first wavelength separation film 33a, is deflected, and enters the image sensor 13a. The image sensor 13a detects the infrared light IRa at the first wavelength and photographs an image of the tissue BT at the first wavelength. The image sensor 13a supplies the data of the photographed image (the photographed image data) to the control apparatus 6.

Among the infrared lights IR radiated from the tissue BT, the infrared light IRb at the second wavelength transmits the first wavelength separation film 33a and the second wavelength separation film 33b and enters the image sensor 13b. The image sensor 13b detects the infrared light IRb at the second wavelength and photographs an image of the tissue BT at the second wavelength. The image sensor 13b supplies the data of the photographed image (the photographed image data) to the control apparatus 6.

Among the infrared lights IR radiated from the tissue BT, the infrared light IRc at the third wavelength is reflected by the second wavelength separation film 33b, is deflected to a side opposite from the infrared light IRa at the first wavelength, and enters the image sensor 13c. The image sensor 13c detects the infrared light IRc at the third wavelength and photographs an image of the tissue BT at the third wavelength. The image sensor 13c supplies the data of the photographed image (the photographed image data) to the control apparatus 6.

The image sensor 13a, the image sensor 13b, and the image sensor 13c are arranged at positions optically conjugated with one another. The image sensor 13a, the image sensor 13b, and the image sensor 13c are arranged such that the optical distances from the photographing optical system 11 become approximately identical.

The image processing system 1 can project the image showing the information on the tissue BT on the tissue BT with the configuration applying the optical detector 3 illustrated in FIG. 16 as well. The optical detector 3 dividedly detects the infrared lights separated by the wavelength separator 33 by the image sensor 13a, the image sensor 13b, and the image sensor 13c, facilitating securing the resolution.

The optical detector 3 may have a configuration that uses a dichroic mirror having the property similar to the first wavelength separation film 33a, and a dichroic mirror having the property similar to the second wavelength separation film 33b instead of the dichroic prism and separates the infrared light by the difference in wavelength. In this case, in the case where any one of optical path lengths of the infrared lights of the infrared light at the first wavelength, the infrared light at the second wavelength, and the infrared light at the third wavelength differs from the optical path lengths of the other infrared lights, the optical path length may be matched using a relay lens or a similar lens.

### (5) Modification of Projector 5

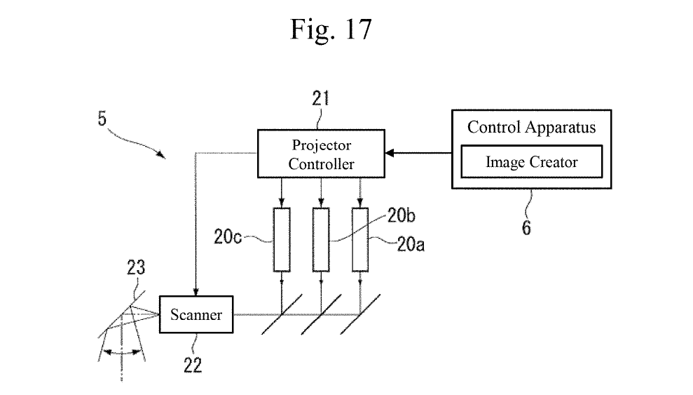

As described above, the projector 5 can project not only the image with a single color but also the image with a plurality of colors. Details of the projector 5 are described here. FIG. 17 is a drawing illustrating the modification of the projector 5. As one example, the projector 5 in FIG. 17 includes a laser light source 20a, a laser light source 20b, and a laser light source 20c irradiating laser beams at wavelengths different from one another.

The laser light source 20a emits laser beam in a red wavelength range. The red wavelength range includes 700 nm, for example, 610 nm or more to 780 nm or less. The laser light source 20b emits laser beam in a green wavelength range. The green wavelength range includes 546.1 nm, for example, 500 nm or more to 570 nm or less. The laser light source 20c emits laser beam in a blue wavelength range. The blue wavelength range includes 435.8 nm, for example, 430 nm or more to 460 nm or less.

In this example, the image creator 4 is configured to form a color image based on an amount of and a proportion of a component as the image projected by the projector 5. For example, the image creator 4 creates green image data such that a green tone value becomes high as the amount of lipid increases. For example, the image creator 4 creates blue image data such that a blue tone value becomes high as the amount of water increases. The control apparatus 6 supplies component image data including the green image data and the blue image data created by the image creator 4 to the projector controller 21.

The projector controller 21 uses the green image data in the component image data supplied from the control apparatus 6 to drive the laser light source 20b. For example, the projector controller 21 increases a current supplied to the laser light source 20b such that optical intensity of green laser beam emitted from the laser light source 20b becomes intense as pixel values specified in the green image data becomes high. Similarly, the projector controller 21 uses the blue image data in the component image data supplied from the control apparatus 6 to drive the laser light source 20c.

The image processing system 1 applying such projector 5 can brightly highlight and display the part where the amount of lipid is large in green and can brightly highlight and display the part where the amount of water is large in blue. The image processing system 1 may brightly display a part where both of the amount of lipid and the amount of water are large in red, or may display an amount of a third substance different from both of the lipid and the water in red.

In FIG. 1 and similar drawings, while the optical detector 3 detects the light passing through the wavelength selection mirror 23 and the projector 5 projects the component image with the light reflected by the wavelength selection mirror 23, the present invention is not limited to such configuration. For example, the optical detector 3 may detect the light reflected by the wavelength selection mirror 23 and the projector 5 may project the component image with the light passing through the wavelength selection mirror 23. The wavelength selection mirror 23 may be a part of the photographing optical system 11 or may be a part of the projection optical system 7. The optical axis 7a of the projection optical system 7 needs not to be coaxial with the optical axis 11a of the photographing optical system 11. One of the plurality of laser light sources 20 may be a laser light source configured to emit the infrared light (the light having the wavelength in the infrared region). With this configuration, for example, even when an infrared camera (an infrared imaging apparatus) not having sensitivity to a wavelength band of the visible light is used for the optical detector 3, this infrared camera can detect the diagram or similar data on the tissue BT projected with the laser light source configured to emit the infrared light. The control apparatus 6 can display the projected diagram or similar data on the display apparatus 31 without performing the above-described image composition.

### (6) Modification of Image Processing System 1

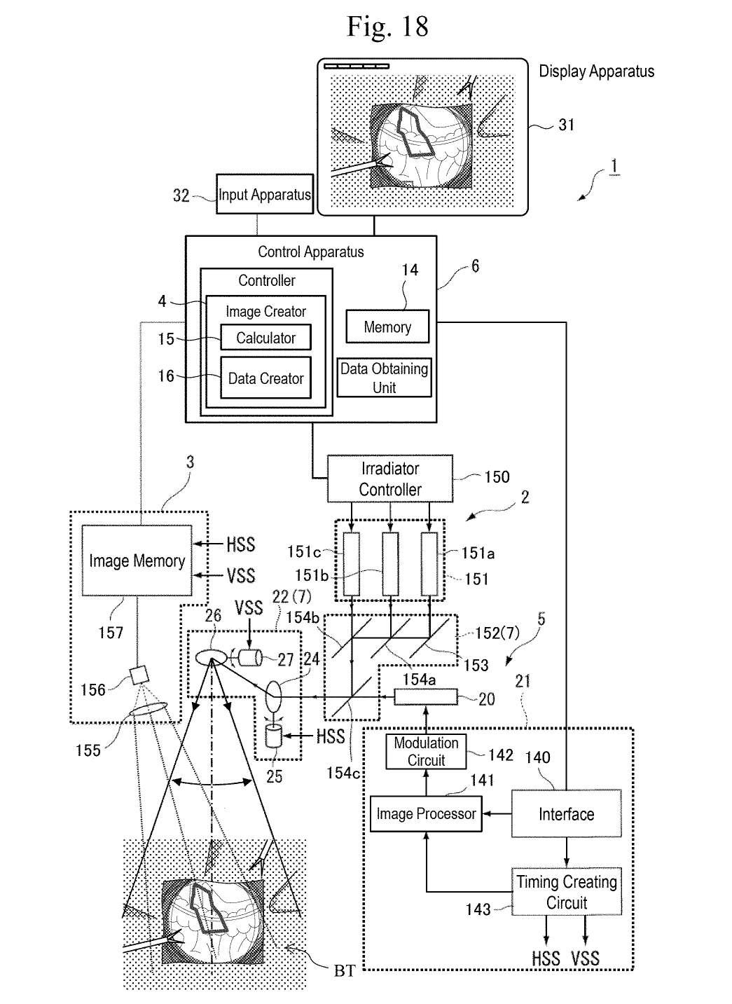

FIG. 18 is a drawing illustrating a configuration of the image processing system 1 according to the modification. Identical reference numerals are given to configurations similar to the above-described embodiment and therefore the following simplifies or omits the explanations.

As one example, the projector controller 21 includes an interface 140, an image processing circuit 141, a modulation circuit 142, and a timing creating circuit 143. The interface 140 receives image data from the control apparatus 6. This image data includes tone data showing the pixel values of the respective pixels and synchronization data specifying, for example, a refresh rate. The interface 140 extracts the tone data from linear data and supplies the tone data to the image processing circuit 141. The interface 140 extracts the synchronization data from the image data and supplies the synchronization data to the timing creating circuit 143.

As one example, the timing creating circuit 143 creates a timing signal indicative of behavior timings of the light source 20 and the scanner 22. The timing creating circuit 143 creates the timing signal according to the resolution of the image, the refresh rate (a frame rate), the scanning method, and a similar specification. Here, it is assumed that the image has a full HD format and, for convenience of explanation, there is no time (a retrace period) from when the drawing of the one horizontal scanning line is ended until the drawing of the next horizontal scanning line starts in the scanning of light.

As one example, the image in the full HD format is a format having the horizontal scanning line on which 1920 pixels are aligned and the format where 1080 horizontal scanning lines are aligned in the vertical scanning direction. To display the image at the refresh rate of 30 Hz, the cycle of the scanning in the vertical scanning direction is about 33 msec ( 1/30 seconds). For example, the second scanning mirror 26 scanning in the vertical scanning direction turns from one end to the other end in a turning range about at 33 msec to scan the one frame image in the vertical scanning direction. A timing creating circuit 143 creates a signal specifying a time at which the second scanning mirror 26 starts drawing the first horizontal scanning line in each frame as a vertical scanning signal VSS. The vertical scanning signal VSS, for example, has a waveform rising at a cycle of about 33 msec.

A drawing period (a lighting period) per horizontal scanning line is, for example, about at 31 microseconds ( 1/30/1080 seconds). For example, the first scanning mirror 24 turns from the one end to the other end in the turning range about 31 microseconds for scanning equivalent to the one horizontal scanning line. The timing creating circuit 143 creates a signal specifying a time at which the first scanning mirror 24 starts scanning the respective horizontal scanning lines as a horizontal scanning signal HSS. The horizontal scanning signal HSS, for example, has a waveform rising at a cycle of about 31 microseconds.

A lighting period per pixel is, for example, about 16 nanoseconds ( 1/30/1080/1920 seconds). For example, by switching the optical intensity of the emitted laser beam at cycles of about 16 nanoseconds according to the pixel values, the light source 20 displays the respective pixels. The timing creating circuit 143 creates a lighting signal to specify a timing at which the light source 20 lights up. The lighting signal, for example, has a waveform rising at a cycle of about 16 nanoseconds.

The timing creating circuit 143 supplies the created horizontal scanning signal HSS to the first driver 25. The first driver 25 drives the first scanning mirror 24 in accordance with the horizontal scanning signal HSS. The timing creating circuit 143 supplies the created vertical scanning signal VSS to the second driver 27. The second driver 27 drives the second scanning mirror 26 in accordance with the vertical scanning signal VSS.

The timing creating circuit 143 supplies the created horizontal scanning signal HSS, vertical scanning signal VSS, and the lighting signals to the image processing circuit 141. The image processing circuit 141 performs various image processes such as a gamma process on the tone data of the image data. The image processing circuit 141 adjusts the tone data based on the timing signal supplied from the timing creating circuit 143 such that the tone data is sequentially output to the modulation circuit 142 at the times matching the scanning method by the scanner 22. The image processing circuit 141, for example, stores the tone data in a frame buffer, reads the pixel values contained in this tone data in the order of the pixels to be displayed, and outputs the pixel values to the modulation circuit 142.

The modulation circuit 142, for example, adjusts the output from the light source 20 such that the intensity of the laser beam emitted from the light source 20 changes as time corresponding to the tone of each pixel. In this modification, the modulation circuit 142 creates a waveform signal in which amplitude changes according to the pixel values and drives the light source 20 by this waveform signal. Accordingly, the current supplied to the light source 20 changes as time according to the pixel values and the optical intensity of the laser beam emitted from the light source 20 changes as time according to the pixel values. Thus, the timing signal created by the timing creating circuit 143 is used to synchronize the light source 20 with the scanner 22.

In this modification, as one example, the irradiator 2 includes an irradiator controller 150, a light source 151, and the projection optical system 7. An irradiator controller 150 controls the lighting and extinction of the light source 151. The light source 151 emits laser beam as detection light. The irradiator 2 deflects the laser beam emitted from a light source 151 in predetermined two directions (for example, a first direction and a second direction) by the projection optical system 7 and scans the tissue BT with the laser beam.

As one example, the light source 151 includes a plurality of laser light sources including a laser light source 151a, a laser light source 151b, and a laser light source 151c. All of the laser light source 151a, the laser light source 151b, and the laser light source 151c include laser elements emitting infrared light, and the wavelengths of the emitted infrared lights are mutually different. The laser light source 151a emits infrared light in a wavelength range containing the first wavelength but not containing the second wavelength and the third wavelength. The laser light source 151b emits infrared light in a wavelength range containing the second wavelength but not containing the first wavelength and the third wavelength. The laser light source 151c emits infrared light in a wavelength range containing the third wavelength but not containing the first wavelength and the second wavelength.

As one example, the irradiator controller 150 supplies currents for driving the laser elements to the respective laser light source 151a, laser light source 151b, and laser light source 151c. The irradiator controller 150 supplies the current to the laser light source 151a to light up the laser light source 151a and stops supplying the current to the laser light source 151a to light out the laser light source 151a. The irradiator controller 150 is controlled by the control apparatus 6 to start or stop supplying the current to the laser light source 151a. For example, the control apparatus 6 controls the timings to light up or light out the laser light source 151a via the irradiator controller 150. Similarly, the irradiator controller 150 lights up or lights out the respective laser light source 151b and laser light source 151c. The control apparatus 6 controls timings to light up or light out the respective laser light source 151b and laser light source 151c.

The projection optical system 7 includes a light guide 152 and the scanner 22. The scanner 22 has a configuration similar to the above-described embodiment, which includes the first scanning mirror 24 and the first driver 25 (the horizontal scanner), and the second scanning mirror 26 and the second driver 27 (the vertical scanner). The light guide 152 guides the detection lights emitted from the respective laser light source 151a, laser light source 151b, and laser light source 151c to the scanner 22 such that the detection lights pass through the optical path identical to the visible light emitted from the light source 20 in the projector 5.

As one example, the light guide 152 includes a mirror 153, a wavelength selection mirror 154a, a wavelength selection mirror 154b, and a wavelength selection mirror 154c. The mirror 153 is arranged at a position where the detection light at the first wavelength emitted from the laser light source 151a enters.

For example, a wavelength selection mirror 154a is arranged at a position where the detection light at the first wavelength reflected by the mirror 153 and the detection light at the second wavelength emitted from the laser light source 151b enter. The wavelength selection mirror 154a has a property where the detection light at the first wavelength transmits and the detection light at the second wavelength is reflected.

The wavelength selection mirror 154b is arranged at a position where the detection light at the first wavelength transmitting the wavelength selection mirror 154a, the detection light at the second wavelength reflected by the wavelength selection mirror 154b, and the detection light at the third wavelength emitted from the laser light source 151c enter. The wavelength selection mirror 154b has a property where the detection light at the first wavelength and the detection light at the second wavelength are reflected and the detection light at the third wavelength transmits.

The wavelength selection mirror 154c is arranged at a position where the detection light at the first wavelength and the detection light at the second wavelength reflected by the wavelength selection mirror 154b, the detection light at the third wavelength transmitting the wavelength selection mirror 154b, and the visible light emitted from the light source 20 enter. The wavelength selection mirror 154c has a property where the detection light at the first wavelength, the detection light at the second wavelength, and the detection light at the third wavelength are reflected and the visible light transmits.

The detection light at the first wavelength, the detection light at the second wavelength, and the detection light at the third wavelength reflected by the wavelength selection mirror 154c and the visible light transmitting the wavelength selection mirror 154c all pass through the identical optical path to enter the first scanning mirror 24 in the scanner 22. The detection light at the first wavelength, the detection light at the second wavelength, and the detection light at the third wavelength that have entered the scanner 22 are each deflected by the scanner 22 similar to the visible light for projecting images. Thus, the irradiator 2 can scan the tissue BT using the scanner 22 with the respective detection light at the first wavelength, detection light at the second wavelength, and detection light at the third wavelength. Accordingly, the image processing system 1 according to the embodiment has a configuration including both a scan type imaging function and a scan type image projection function.

In this modification, the optical detector 3 detects the light radiated from the tissue BT laser-scanned by the irradiator 2. The optical detector 3 associates the optical intensity of the detected light with the position information of the laser beam irradiated from the irradiator 2 to detect a space distribution of the optical intensity of the light radiated from the tissue BT in the range in which the irradiator 2 performs the scanning with the laser beam. As one example, the optical detector 3 includes a condenser lens 155, an optical sensor 156, and an image memory 157.

The optical sensor 156 includes a photodiode such as a silicon PIN photodiode and a GaAs photodiode. The condenser lens 155 condenses at least a part of the light radiated from the tissue BT to the photodiode of the optical sensor 156. The condenser lens 155 needs not to form the image of the tissue BT (the irradiated region with the detection light).

The image memory 157 stores a digital signal output from the optical sensor 156. The projector controller 21 supplies the image memory 157 with the horizontal scanning signal HSS and the vertical scanning signal VSS. The image memory 157 uses the horizontal scanning signal HSS and the vertical scanning signal VSS to convert the signal output from the optical sensor 156 into data in the image format. For example, the image memory 157 converts a detection signal output from the optical sensor 156 in a period from rising to falling of the vertical scanning signal VSS into one frame image data. The optical detector 3 supplies the detected image data to the control apparatus 6.

The control apparatus 6 controls the wavelength of the detection light irradiated by the irradiator 2. The control apparatus 6 controls the irradiator controller 150 to control the wavelength of the detection light emitted from the light source 151. The control apparatus 6 supplies control signals to specify timings to light up or light out the laser light source 151a, the laser light source 151b, and the laser light source 151c to the irradiator controller 150. The irradiator controller 150 selectively lights up the laser light source 151a, which emits the light at the first wavelength, the laser light source 151b, which emits the light at the second wavelength, and the laser light source 151c, which emits the light at the third wavelength, based on the control signals supplied from the control apparatus 6.

For example, the control apparatus 6 causes the optical detector 3 to detect the light radiated from the tissue BT in a first period during which the irradiator 2 irradiates the light at the first wavelength. The control apparatus 6 causes the optical detector 3 to detect the light radiated from the tissue BT in a second period during which the irradiator 2 irradiates the light at the second wavelength. The control apparatus 6 causes the optical detector 3 to detect the light radiated from the tissue BT in a third period during which the irradiator 2 irradiates the light at the third wavelength. The control apparatus 6 controls the optical detector 3 to cause the optical detector 3 to separately output the detection result by the optical detector 3 in the first period, the detection result by the optical detector 3 in the second period, and the detection result by the optical detector 3 in the third period to the image creator 4.

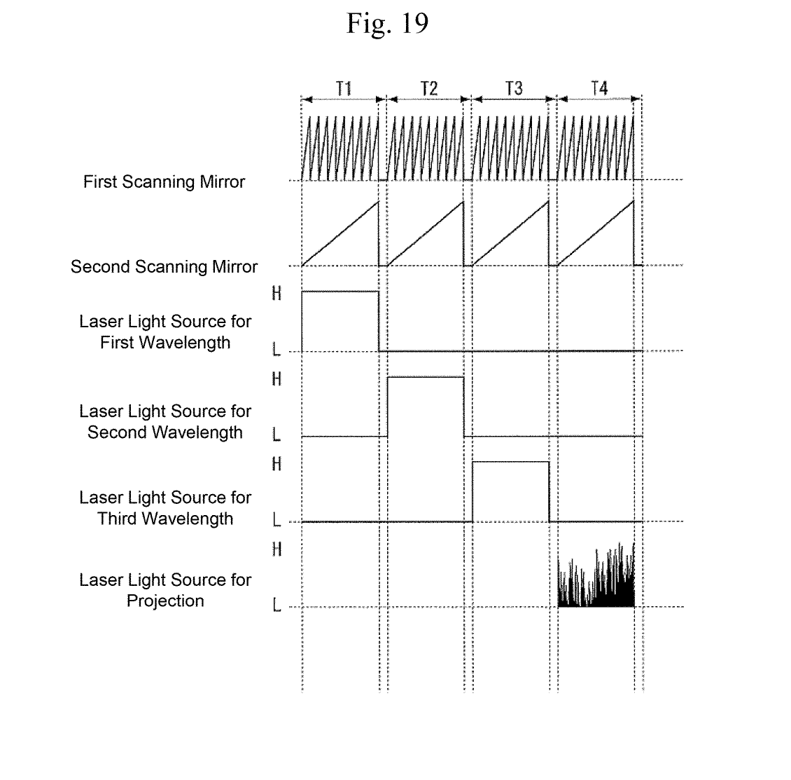

FIG. 19 is a timing chart illustrating one example of behaviors of the irradiator 2 and the projector 5. FIG. 19 illustrates an angular position of the first scanning mirror 24, an angular position of the second scanning mirror 26, and an electric power supplied to each light source. A first period T1 is equivalent to a display period of one frame, and the length is around 1/30 seconds at the refresh rate of 30 Hz. The same applies to a second period T2, a third period T3, and a fourth period T4.

In the first period T1, the control apparatus 6 lights up the laser light source 151a for the first wavelength. In the first period T1, the control apparatus 6 lights out the laser light source 151b for the second wavelength and the laser light source 151c for the third wavelength.

In the first period T1, the first scanning mirror 24 and the second scanning mirror 26 behave under conditions identical to the conditions when the projector 5 projects the image. In the first period T1, the first scanning mirror 24 repeatedly turns from the one end to the other end in the turning range by the number of horizontal scanning lines. A unit waveform from the rising until the next rising at the angular position of the first scanning mirror 24 is equivalent to the angular position during which the one horizontal scanning line is scanned. For example, with the image projected by the projector 5 in the full HD format, the first period T1 includes the unit waveform at the angular position of the first scanning mirror 24 by 1080 cycles. In the first period T1, the second scanning mirror 26 once turns from the one end to the other end in the turning range.

By such behavior by the scanner 22, the laser beam at the first wavelength emitted from the laser light source 151a scans the entire region in the scanning range on the tissue BT. The control apparatus 6 obtains first detection image data equivalent to the result detected by the optical detector 3 in the first period T1 from the optical detector 3.

In the second period T2, the control apparatus 6 lights up the laser light source 151b for the second wavelength. In the second period T2, the control apparatus 6 lights out a laser light source 151a for the first wavelength and the laser light source 151c for the third wavelength. In the second period T2, the first scanning mirror 24 and the second scanning mirror 26 behave similar to the first scanning mirror 24 and the second scanning mirror 26 in the first period T1. Accordingly, the laser beam at the second wavelength emitted from this laser light source 151b scans the entire region in the scanning range on the tissue BT. The control apparatus 6 obtains second detection image data equivalent to the result detected by the optical detector 3 in the second period T2 from the optical detector 3.

In the third period T3, the control apparatus 6 lights up the laser light source 151c for the third wavelength. In the third period T3, the control apparatus 6 lights out the laser light source 151a for the first wavelength and the laser light source 151b for the second wavelength. In the third period T3, the first scanning mirror 24 and the second scanning mirror 26 behave similar to the first scanning mirror 24 and the second scanning mirror 26 in the first period T 1. Accordingly, the laser beam at the third wavelength emitted from this laser light source 151c scans the entire region in the scanning range on the tissue BT. The control apparatus 6 obtains third detection image data equivalent to the result detected by the optical detector 3 in the third period T3 from the optical detector 3.

The image creator 4 illustrated in FIG. 18 creates the component image using the first detection image data, the second detection image data, and the third detection image data and supplies the component image data to the projector 5. The image creator 4 uses the detection image data instead of photographed image data described in the above-described embodiment to create the component image. For example, the calculator 15 uses the time change in the optical intensity of the light detected by the optical detector 3 to calculate the information on the component of the tissue BT.

In the fourth period T4, the projector controller 21 illustrated in FIG. 18 uses the component image data supplied from the control apparatus 6 to supply a driving electric power wave where the amplitude changes as time according to the pixel values to the light source 20 for projection and control the scanner 22. Thus, the projector 5 projects the component image on the tissue BT in the fourth period T4.

The image processing system 1 according to this modification detects the light radiated from the tissue BT by the optical sensor 156 while laser-scanning the tissue BT with the detection light to obtain the detection image data equivalent to the photographed image data of the tissue BT. The optical sensor 156 may be one where the number of pixels is smaller than that of the image sensor. Therefore, downsizing, weight reduction, low cost, and a similar feature are possible with the image processing system 1. A light-receiving area of the optical sensor 156 is easily configured to be larger than a light-receiving area of one pixel of the image sensor, thereby ensuring enhancing the detection accuracy of the optical detector 3.

In this modification, the irradiator 2 includes the plurality of light sources that emit the lights at wavelengths different from one another, temporally switches the light source to be lit up among the plurality of light sources, and irradiates the detection light. Therefore, compared with the configuration where the detection light at a broad wavelength is irradiated, this allows reducing the light at the wavelength not detected by the optical detector 3. Therefore, for example, energy per unit time given to the tissue BT by the detection light can be reduced, thereby ensuring reducing a temperature rise of the tissue BT by the detection light L1. This also ensures configuring the intense optical intensity of the detection light without increasing the energy per unit time given to the tissue BT by the detection light, ensuring enhancing the detection accuracy of the optical detector 3.

In FIG. 19, the first period T1, the second period T2, and the third period T3 are the irradiation periods during which the irradiator 2 irradiates the detection light and are also the detection periods during which the optical detector 3 detects the light radiated from the tissue BT. The projector 5 does not project the images in at least a part of the irradiation periods and the detection periods. Therefore, the projector 5 can display the image such that the projected image is visually perceived flickery. Therefore, the user easily identifies the component image or similar image from the tissue BT.

The projector 5 may project the image in at least a part of the irradiation periods and the detection periods. For example, the image processing system 1 may create a first component image using the result detected by the optical detector 3 in a first detection period and project the first component image on the tissue BT in at least a part of a second detection period after the first detection period. For example, while the projector 5 projects the image, the irradiator 2 may irradiate the detection light and the optical detector 3 may detect the light. While the projector 5 displays an image of a first frame, the image creator 4 may create image data of a second frame to be projected after the first frame. The image creator 4 may create the image data of the second frame using the result detected by the optical detector 3 while the image of the first frame is displayed. The projector 5 may project the image of the second frame subsequent to the above-described image of the first frame.

While in this modification, the irradiator 2 alternatively switches the light source to be lit up among the plurality of light sources to irradiate the detection light, the two or more light sources among the plurality of light sources may be lit up concurrently to irradiate the detection light. For example, the irradiator controller 150 may control the light source 151 such that all of the laser light source 151a, the laser light source 151b, and the laser light source 151c are lit up. In this case, like FIG. 16, the optical detector 3 may perform wavelength separation on the light radiated from the tissue BT and detect the light by each wavelength.

### (7) Development to Projecting Apparatus

While in the configurations illustrated in FIG. 1 and the similar drawings, the projector 5 and the control apparatus 6 are illustrated as the independent processors (configurations) and the functions are explained, for example, the functions of the projector 5 and the control apparatus 6 may be configured integrally and provided as a projecting apparatus, and, for example, the functions of the projector 5 and the functions of the control apparatus 6 other than the image creator 4 may be configured integrally and provided as a projecting apparatus.

In the case where the functions of the projector 5 and the functions of the control apparatus 6 other than the image creator 4 are configured integrally, the projecting apparatus includes, for example, a projector that irradiates a biological tissue with visible light, and a controller that controls a projection behavior by the projector such that contents of an input are reflected to the biological tissue in response to the input to the display apparatus (displays an image of the biological tissue created using a detection result by an optical detector that detects light radiated from the biological tissue irradiated with infrared light) 31.

In the case where the functions of the projector 5 and the control apparatus 6 are integrally configured, the projecting apparatus includes, for example, a projector that irradiates the biological tissue with visible light and projects a diagram on the biological tissue and a controller that analyzes a detection result by an optical detector, which detects light radiated from the biological tissue irradiated with infrared light, to identify the affected part in the biological tissue, transmits information on this affected part to a display apparatus as an analysis result, superimposes and displays the information on the affected part with an image created based on the detection result by the optical detector in the display apparatus, and controls the projection of the diagram on the affected part in the biological tissue by the projector based on the analysis result.

### (8) Application to Fluorescent Observation

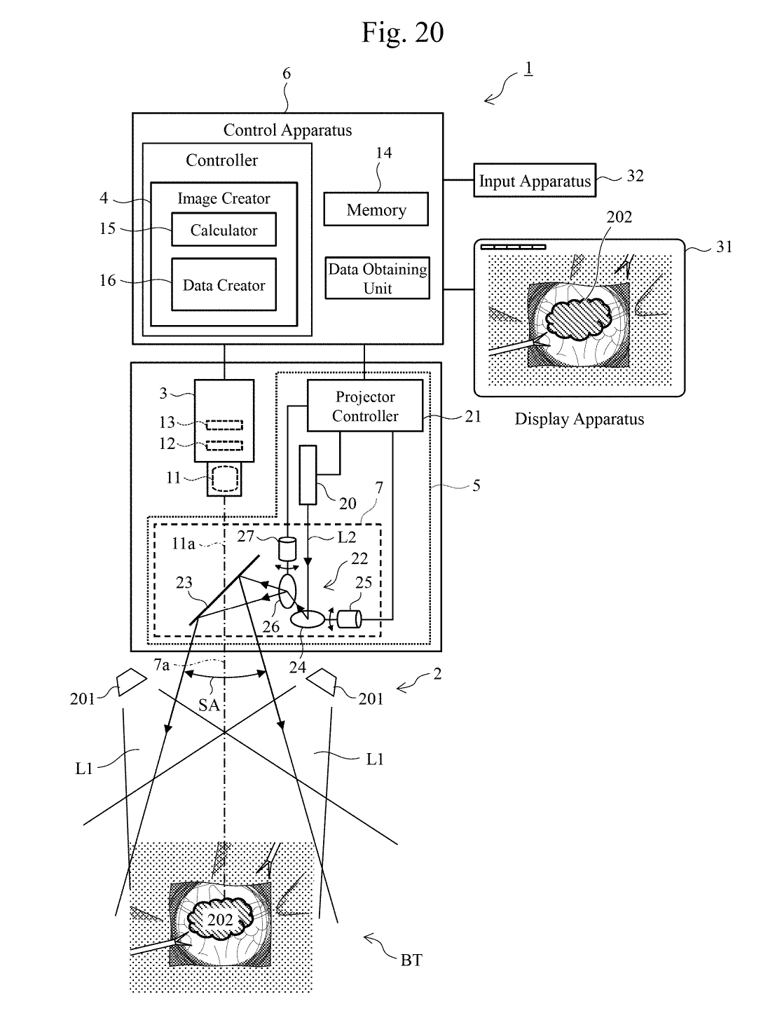

As an application example to the fluorescent observation, the image processing system 1 according to the embodiment can inject solution containing a biocompatible fluorescent agent such as indocyanine green (ICG) into a living body (for example, the tissue BT) and observe the tissue BT using a property of this fluorescent agent gathering at a specific tissue. FIG. 20 is a drawing describing the overview of these functions and configuration. The configuration of the image processing system 1 in FIG. 20 is similar to FIG. 1 and therefore the following omits the detailed explanation of the configuration.

In the fluorescent observation, for example, the indocyanine green (ICG) is injected into the tissue BT. For example, in the case where the injected ICG gathers at a specific region (for example, a tumor) in the tissue BT, irradiating the tissue BT with excitation light at a wavelength of 760 nm from a light source 201 according to the embodiment generates fluorescent at a wavelength of 830 nm at the specific region. For example, the light source 201 in FIG. 20 is a LED light source emitting light at the wavelength of 760 nm and is controlled by the controller. For example, the optical detector 3 is configured so as to have detection sensitivity to fluorescence at the wavelength of 830 nm radiated from the tissue BT. For example, when the optical detector 3 images the tissue BT while the control by the controller emits the light source 201, the image processing system 1 can obtain the image data with high illuminance at a specific region (site) 202 where the ICG gathers much in the tissue BT. The image processing system 1 projects an image (a tone image) created by performing gradation conversion on this obtained image data with high illuminance by a predetermined threshold (for example, binarization) on the tissue BT to allow the user to observe a verification state (or a collected state) of the ICG by naked eyes.

For example, the above-described Function 2 and Function 3 may be executed in a fluorescent observation mode. For example, to execute Function 2 with the image processing system 1 illustrated in FIG. 20, for example, when the user (for example, the operator) uses the input apparatus to input (draw) the marking (any diagram) to the specific region 202 while the photographed image of the tissue BT is displayed in the screen of the display apparatus 31, the control apparatus 6 controls the projector 5 to project a diagram identical to the input marking on a position on the tissue BT (the specific region) identical to the position to which the marking has been input with the visible light. By thus projecting the diagram identical to the diagram input in the display screen on the identical position on the tissue BT, the user can appropriately and easily treat (such as the surgery and the examination) the site on the tissue BT (for example, the specific region) corresponding to the site confirmed in the screen in the fluorescent observation as well.

### (9) Application to Multi-Modality

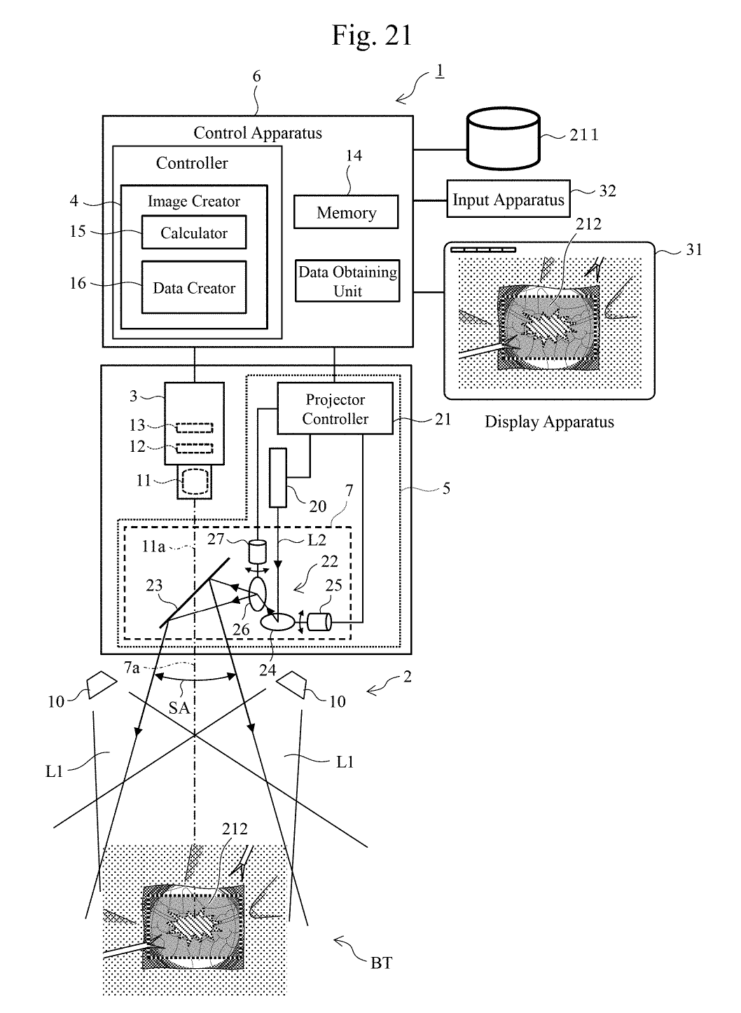

As the application example to the multi-modality, the image processing system 1 according to the embodiment may include a function to project information (for example, an image) input from another image diagnostic apparatus such as an MRI and an ultrasonic image diagnostic apparatus on the tissue BT, in addition to the function to project the above-described input diagram or similar data on the tissue BT. FIG. 21 is a drawing describing the overview of these functions and configuration. The configuration of the image processing system 1 in FIG. 21 is similar to FIG. 1 and therefore the following omits the detailed explanation of the configuration.

A storage apparatus 211 is a memory that stores information (for example, an image 212 of the tissue BT) on the tissue BT preliminary obtained (collected) by another (external) image diagnostic apparatus (for example, the MRI and the ultrasonic image diagnostic apparatus). For example, this image 212 is read by the control apparatus, displayed on the display apparatus 31, and is projected on the tissue BT via the projector. For example, the operating person can arbitrarily set color, position, size, and similar specification of the image 212 with the input apparatus 32 at this time. Since such configuration allows the operating person to directly visually perceive the tissue BT and the image 212, a predetermined treatment can be performed based on the image highlighted by the other image diagnostic apparatus. For example, the image processing system 1 may superimpose a near-infrared image photographed by an imaging apparatus with the image 212 and display the image on the display apparatus or may superimpose the near-infrared image photographed by the imaging apparatus with the image 212 and project the image on the tissue BT. For example, the storage apparatus 211 may be a cloud coupled with, for example, the Internet.

### Another One Embodiment

(i) In addition to a process damaging the tissue BT like general surgeries, the image processing system 1 is applicable to a medical treatment application, an examination application, a survey application, and a similar application such as various processes not damaging the tissue BT. The image processing system 1 is usable for, for example, a blood sampling, a pathological anatomy, a pathological diagnosis (including a rapid intraoperative diagnosis), laboratory study such as an antemortem examination (a biopsy), and sampling assistance for biomarker search. For example, the tissue BT may be a human tissue (for example, a body tissue) or maybe a tissue for an organism other than the human. For example, the tissue BT may be a tissue cut out from the organism or may be a tissue attached to the organism. Additionally, for example, the tissue BT may be a tissue (for example, a biological tissue) of the living organism or may be a tissue of an organism (a cadaver) after death. The tissue BT may be an object extracted from an organism. For example, the tissue BT may include any organ of the organism, may include a skin, or may include an internal organ inside the skin or a similar organ. Therefore, the tissue BT can be referred to as a biological tissue.

(ii) As illustrated in FIG. 1, in the image processing system 1 according to this embodiment, all of the configurations may be arranged at the identical site. Meanwhile, at least the irradiator (the irradiating apparatus) 2, the optical detector (the light detecting apparatus such as an infrared light camera) 3, and the projector (the projecting apparatus) 5 only need to be installed at a location to process the tissue BT (as one example, the operating room and an examination room), and the control apparatus 6, the display apparatus 31, and the input apparatus 32 may be remotely installed. In this case, the control apparatus 6 only needs to be coupled to the optical detector 3 and the projector 5 over a network. The display apparatus 31 and the input apparatus 32 may be installed at yet other positions over a network.

(iii) While the image (the photographed image) of the biological tissue (the tissue BT) is displayed in the screen of the display apparatus, in the case where the input is performed on this image, the image processing system according to this embodiment reflects these contents of the input and controls the projection behavior by the projector (the projecting apparatus). The position where the contents of the input are reflected on the biological tissue corresponds to the input position on the display image. Here, the input includes a diagram drawn on the screen of the display apparatus, input characters and signs, a selected diagram or similar data in display, or similar data. Thus, the action to the image displayed in the screen can be reflected to the actual biological tissue as it is. Accordingly, the operating person can appropriately and easily confirm and identify the target site for the medical practice, an examination action, or a similar action and therefore can smoothly advance various processes. This embodiment allows sufficiently and appropriately assisting the actions such as the medical practice and the examination action.

This image processing system includes the photographing optical system and the projection optical system configured as the optical systems coaxial with one another. This eliminates the need for positioning the screen of the display apparatus and the tissue BT, thereby allowing a reduction in load in the image processing.

This image processing system may analyze the detection result by the optical detector (as one example, the infrared sensor) and display this result of analysis (this component image) on the screen of the display apparatus together with the photographed image. This configuration allows the confirmation of the site (the component image) identified as the affected part on the screen as well, not only on the biological tissue (the tissue BT).

This image processing system identifies the plurality of candidates for affected part (the component images at a plurality of parts) through analysis and displays the information on these plurality of candidates for affected part on the screen of the display apparatus. Then, the image processing system replies to at least one of the selection inputs in the information on the plurality of candidates for affected part and reflects the selection input to the projection by the projecting apparatus (the projector). Thus, the image processing system 1 displays the sites possibly the affected part based on the result of component analysis on the screen. Accordingly, the image processing system 1 can submit the specific regions suspected as the affected site to be treated, easily identify the site of the affected part with conviction among the specific regions, and appropriately execute the action such as the medical practice and the examination action.

This image processing system may execute the photographing behavior (the detection behavior by the optical detector (as one example, the infrared sensor)) and the projection behavior of the contents of the input by the projecting apparatus time-divisionally. This ensures avoiding the image projected on the biological tissue (the tissue BT) to adversely affect the photographing behavior when the photographing behavior and the projection behavior are simultaneously performed in real-time.

This image processing system may irradiate the biological tissue (the tissue BT) from the projector (the projecting apparatus) while switching the wavelength of the visible light in units of predetermined time intervals or may irradiate the biological tissue while flashing this visible light. This highlight display of the projection image ensures ease of seeing the image projected on the biological tissue.

The operating person (such as the operator and the examiner) wears the shutter glasses (as one example, the liquid crystal shutter glasses), and this image processing system causes the left eye shutter and the right eye shutter of these glasses to open/close in alternation at every predetermined time interval. The image processing system controls each of the LED light source and the projecting apparatus such that the lighting of the LED light source illuminating the biological tissue (the tissue BT) and the irradiation of the visible light from the projector (the projecting apparatus) are performed in alternation. The image processing system matches the opening/closing timings of any one of the left eye shutter and the right eye shutter (for example, the left eye shutter) with the ON/OFF timings of the LED light source. Meanwhile, the image processing system matches the opening/closing timings of the shutter (for example, the right eye shutter) other than the shutter that has been matched with the ON/OFF timings of the LED light source with the ON/OFF timings of the irradiation of the visible light. Such highlight display of the projection image allows the operating person to see the image projected on the biological tissue free from problem even if the biological tissue is illuminated considerably brightly and therefore the visible light projected on the biological tissue is hard to be seen usually. This image processing system may employ an image sensor that detects three-dimensional images and a display apparatus that displays the three-dimensional images. Accordingly, the operating person can three-dimensionally confirm the affected site in the display screen and therefore can confirm the position of the affected site more accurately.

(iv) This image processing system analyzes the detection result by the optical detector (as one example, the infrared sensor) to identify the affected part in the biological tissue, superimposes and displays the information (for example, the outline surrounding the affected part) on the affected part as the analysis result with the photographed image of the entire biological tissue (the tissue BT), and irradiates and projects the information on the affected part (the outline surrounding the affected part) on the biological tissue (the tissue BT). Thus, in the case where the affected site is difficult to be identified only by simply displaying the component image in the screen and projecting the component image on the biological tissue, this image processing system can provide the information with which the affected site can be identified with more certainty.

(v) The present invention can also be achieved by program codes of software achieving the functions of the embodiments. In this case, a storage medium recording the program codes are provided to the system or the apparatus, and a computer (or a CPU and an MPU) in the system or the apparatus reads the program codes stored in the storage medium. In this case, the program codes themselves read from the storage medium achieve the above-described functions of the embodiments. The program codes themselves and the storage medium storing the program codes configure the present invention. As examples of the storage medium supplying such program codes, a flexible disk, a CD-ROM, a DVD-ROM, a hard disk, an optical disk, a magneto-optical disk, a CD-R, a magnetic tape, a non-volatile memory card, and a ROM are used.

An operating system (OS) operating on the computer or a similar system may perform a part of or all of the actual processes based on instructions from the program codes, and the above-described functions of the embodiments may be achieved by the processes. Furthermore, after the program codes read from the storage medium are written to a memory in the computer, the CPU in the computer or a similar system may perform a part of or all of the actual processes based on the instructions from the program codes, and the above-described functions of the embodiments may be achieved by the processes.

Furthermore, the program codes of the software, which achieve the functions of the embodiments, may be distributed over a network, storage means such as a hard disk and a memory in the system or the apparatus or the storage medium such as the CD-RW and the CD-R may store the program codes, and the computer (or the CPU and the MPU) in the system or the apparatus may read and execute the program codes stored in this storage means and this storage medium for use.

According to this embodiment, there is provided the image processing system that includes the infrared light irradiating apparatus, the optical detector, the control apparatus, the display apparatus, and the projecting apparatus. The infrared light irradiating apparatus is configured to irradiate the biological tissue with the infrared light. The optical detector is configured to detect the light radiated from the biological tissue irradiated with the infrared light. The control apparatus is configured to create the image of the biological tissue using the detection result by the optical detector. The display apparatus is configured to display the created image. The projecting apparatus is configured to irradiate the biological tissue with the first light. The control apparatus is configured to control the irradiation with the first light by the projecting apparatus such that the contents of the input are reflected to the biological tissue in response to the input to the display apparatus configured to display the image of the biological tissue.

According to this embodiment, there is provided the image processing system that includes the infrared light irradiating apparatus, the optical detector, the control apparatus, the display apparatus, and the projecting apparatus. The infrared light irradiating apparatus is configured to irradiate the biological tissue with the infrared light. The optical detector is configured to detect the light radiated from the biological tissue irradiated with the infrared light. The control apparatus is configured to create the image of the biological tissue using the detection result by the optical detector. The display apparatus is configured to display the created image. The projecting apparatus is configured to project the diagram to the biological tissue. The control apparatus is configured to analyze the detection result by the optical detector to identify the affected part in the biological tissue, transmit the information on this affected part to the display apparatus as the analysis result, superimpose and display the information on the affected part with the created image in the display apparatus, and control the projection of the diagram on the affected part in the biological tissue by the projecting apparatus based on the analysis result.

According to this embodiment, there is provided the image processing apparatus that includes the controller. The controller is configured to create the image of the biological tissue using the detection result by the optical detector. The optical detector is configured to detect the light radiated from the biological tissue irradiated with the infrared light. The controller is configured to transmit this created image to the display apparatus such that the display apparatus displays the created image. This controller is configured to control the irradiation by the projector such that the projector irradiates the biological tissue with the first light to reflect the contents of the input to the biological tissue in response to the input to the display apparatus configured to display the image of the biological tissue.

According to this embodiment, there is provided the image processing apparatus that includes the controller. The controller is configured to create the image of the biological tissue using the detection result by the optical detector. The optical detector is configured to detect the light radiated from the biological tissue irradiated with the infrared light. The controller is configured to transmit this created image to the display apparatus such that the display apparatus displays the created image. This controller is configured to analyze the detection result by the optical detector to identify the affected part in the biological tissue, transmit the information on this affected part to the display apparatus as the analysis result, superimpose and display the information on the affected part with the created image in the display apparatus, and control the projection of the diagram on the affected part in the biological tissue by the projector configured to irradiate the biological tissue with the light based on the analysis result.

According to the embodiment, there is provided the projection method that includes the irradiating, the detecting, the creating, the displaying, and the controlling. The irradiating irradiates the biological tissue with the infrared light. The detecting detects the light radiated from the biological tissue irradiated with the infrared light. The creating creates the image of the biological tissue using the detection result of the light radiated from the biological tissue. The displaying displays the created image of the biological tissue on the display apparatus. The controlling controls the irradiation of the first light by the projecting apparatus such that the contents of the input are reflected to the biological tissue in response to the input to the display apparatus configured to display the image of the biological tissue.

According to the embodiment, there is provided the projection method that includes the irradiating, the detecting, the creating, the displaying, the analyzing, and the controlling. The irradiating irradiates the biological tissue with the infrared light. The detecting detects the light radiated from the biological tissue irradiated with the infrared light. The creating creates the image of the biological tissue using the detection result of the light radiated from the biological tissue. The displaying displays the created image of the biological tissue on the display apparatus. The analyzing analyzes the detection result to identify an affected part in the biological tissue. The analyzing transmits the information on this affected part to the display apparatus as the analysis result, superimposes the information on the affected part on the created image and causes the display apparatus to display the superimposed image. The controlling controls the projection of the diagram to the affected part in the biological tissue by the projecting apparatus based on the analysis result.

According to the embodiment, there is provided the projecting apparatus that includes the projector and the controller. The projector is configured to irradiate the biological tissue with the first light. The controller is configured to control the irradiation with the first light by the projector such that the contents of the input are reflected to the biological tissue in response to the input to the display apparatus configured to display the image of the biological tissue. The image is created using the detection result by the optical detector. The optical detector is configured to detect the light radiated from the biological tissue irradiated with the infrared light.

According to the embodiment, there is provided the projecting apparatus that includes the projector and the controller. The projector is configured to project the diagram to the biological tissue. The controller is configured to analyze the detection result by the optical detector configured to detect the light radiated from the biological tissue irradiated with infrared light to identify the affected part in the biological tissue, transmit the information on this affected part to the display apparatus as the analysis result, superimpose and display the information on the affected part with the image created based on the detection result by the optical detector in the display apparatus, and control the projection of the diagram on the affected part in the biological tissue by the projector based on the analysis result.

The processes and techniques described here are not essentially related to any particular apparatus, and can be mounted by any suitable combination of components. Furthermore, general-purpose, various types of apparatuses can be used in accordance with the method described here. For executing steps of the method described here, it may be beneficial to construct a dedicated apparatus. Further, various inventions can be made by properly combining the plurality of constituents disclosed in the embodiments. For example, some constituents may be omitted from all of the constituents shown in the embodiments. Moreover, components in different embodiments may be suitably combined together.

Other implementation of the present invention will be made apparent for those having ordinary knowledge in the technical field from the examination of the specification and the embodiments of the present invention disclosed herein. The various forms and/or components of the explained embodiments can be used independently or in any combination.

### REFERENCE SIGNS LIST

1 Image processing system
2 Irradiator
3 Optical detector
4 Image creator
5 Projector
6 Control apparatus
7 Projection optical system
11 Photographing optical system
15 Calculator
16 Data creator
22 Scanner
31 Display apparatus
32 Input apparatus

1. An image processing system comprising:
an infrared light irradiating apparatus configured to irradiate a biological tissue with infrared light;
an optical detector configured to detect detection light radiated from the biological tissue irradiated with the infrared light;
a display apparatus configured to display an image of the biological tissue, the image being created using a detection result by the optical detector;
a projecting apparatus configured to irradiate the biological tissue with first light; and
a control apparatus configured to control the irradiation with the first light by the projecting apparatus such that contents of an input are reflected to the biological tissue based on the input to the display apparatus configured to display the image of the biological tissue.

2. The image processing system according to claim 1,
wherein the optical detector includes an optical system, the optical system configuring optical systems with an optical system of the projecting apparatus coaxial with one another.

3. The image processing system according to claim 1,
wherein the control apparatus is configured to control the projecting apparatus such that the projecting apparatus irradiates a position in the biological tissue corresponding to an input position of the input in the image with the contents of the input based on the input to the image of the biological tissue displayed on the display apparatus.

4. The image processing system according to claim 1,
wherein the control apparatus is configured to analyze the detection result by the optical detector, the control apparatus being configured to transmit a result of the analysis to the display apparatus, and
the display apparatus is configured to display the result of the analysis together with the image.

5. The image processing system according to claim 1,
wherein the control apparatus is configured to analyze the detection result by the optical detector to identify a plurality of candidates for affected part, the control apparatus being configured to transmit information on the plurality of candidates for affected part to the display apparatus,
the display apparatus is configured to display the information on the plurality of candidates for affected part together with the image, and
the control apparatus is further configured to control the projecting apparatus based on at least one selection input in the information on the plurality of candidates for affected part, the control apparatus being configured to reflect the selection input to the irradiation of the contents of the input by the projecting apparatus.

6. The image processing system according to claim 4,
wherein the control apparatus is configured to analyze the detection result by the optical detector, the control apparatus being configured to transmit data showing a water or a lipid at the biological tissue as a result of the analysis to the display apparatus.

7. The image processing system according to claim 1,
wherein the control apparatus is configured to time-divisionally execute a detection behavior by the optical detector and a light irradiation behavior of the contents of the input by the projecting apparatus.

8. The image processing system according to claim 1,
wherein the control apparatus is configured to control the projecting apparatus such that the projecting apparatus irradiates the biological tissue with the first light while the projecting apparatus switches a wavelength of the first light in units of predetermined time intervals or such that the projecting apparatus irradiates the biological tissue with the first light while the projecting apparatus flashes the first light.

9. The image processing system according to claim 1, further comprising
an eyesight restricting apparatus,
wherein the biological tissue is irradiated by a LED light source,
the eyesight restricting apparatus is configured to control opening/closing of an eyesight of a left eye and an eyesight of a right eye of a wearer of the eyesight restricting apparatus in alternation at every predetermined time interval,
the control apparatus is configured to control each of the LED light source and the projecting apparatus such that a lighting of the LED light source and the irradiation with the first light are performed in alternation, and
the control apparatus is configured to match opening/closing timings of any one of the eyesight of the left eye and the eyesight of the right eye with ON/OFF timings of the LED light source, the control apparatus being configured to match opening/closing timings of an eyesight other than the eyesight that has been matched with the ON/OFF timings of the LED light source with ON/OFF timings of the irradiation with the first light.

10. The image processing system according to claim 1,
wherein the optical detector is an image sensor configured to detect a three-dimensional image, and
the display apparatus is an apparatus configured to display a three-dimensional image.

11. An image processing system comprising:
an infrared light irradiating apparatus configured to irradiate a biological tissue with infrared light;
an optical detector configured to detect detection light radiated from the biological tissue irradiated with the infrared light;
a display apparatus configured to display an image of the biological tissue, the image being created using a detection result by the optical detector;
a projecting apparatus configured to irradiate the biological tissue with light; and
a control apparatus configured to analyze the detection result by the optical detector to identify an affected part in the biological tissue, the control apparatus being configured to superimpose information on the affected part on the image of the biological tissue and cause the display apparatus to display the superimposed image, the control apparatus being configured to control the irradiation of the information on the affected part to the biological tissue by the projecting apparatus.

12. The image processing system according to claim 11,
wherein the information on the affected part includes position information of the affected part obtained through the analysis of the detection result.

13. The image processing system according to claim 11,
wherein the optical detector includes an optical system, the optical system configuring optical systems with an optical system of the projecting apparatus coaxial with one another.

14. The image processing system according to claim 11,
wherein the control apparatus is configured to control a detection behavior by the optical detector and a light irradiation behavior by the projecting apparatus to be time-divisionally executed.

15. The image processing system according to claim 11,
wherein the projecting apparatus is configured to irradiate the information on the affected part to the biological tissue using first light, and
the control apparatus is configured to control the projecting apparatus such that the projecting apparatus irradiates the biological tissue with the first light while the projecting apparatus switches a wavelength of the first light in units of predetermined time intervals or such that the projecting apparatus irradiates the biological tissue with the first light while the projecting apparatus flashes the first light.

16. The image processing system according to claim 11, further comprising
an eyesight restricting apparatus,
wherein the projecting apparatus is configured to irradiate the biological tissue with the information on the affected part using first light,
the biological tissue is irradiated by a LED light source,
the eyesight restricting apparatus is configured to control opening/closing of an eyesight of a left eye and an eyesight of a right eye of a wearer of the eyesight restricting apparatus in alternation at every predetermined time interval,
the control apparatus is configured to control each of the LED light source and the projecting apparatus such that a lighting of the LED light source and the irradiation with the first light are performed in alternation, and
the control apparatus is configured to match opening/closing timings of any one of the eyesight of the left eye and the eyesight of the right eye with ON/OFF timings of the LED light source, the control apparatus being configured to match opening/closing timings of an eyesight other than the eyesight that has been matched with the ON/OFF timings of the LED light source with ON/OFF timings of the irradiation with the first light.

17. The image processing system according to claim 11,
wherein the display apparatus is an apparatus configured to display a three-dimensional image.

18. The image processing system according to claim 11,
wherein the control apparatus is configured to analyze the detection result by the optical detector, the control apparatus being configured to superimpose information on a plurality of the affected parts in the biological tissue on the image of the biological tissue and cause the display apparatus to display the superimposed image.

19. An image processing system comprising
a controller configured to create an image of a biological tissue using a detection result by an optical detector, the optical detector being configured to detect detection light radiated from the biological tissue irradiated with infrared light, the controller being configured to transmit the created image to a display apparatus such that the display apparatus displays the created image,
wherein the controller is configured to control an irradiation by a projector such that the projector irradiates the biological tissue with light to reflect contents of an input to the biological tissue based on the input to the display apparatus configured to display the image of the biological tissue.

20. An image processing apparatus comprising
a controller configured to create an image of a biological tissue using a detection result by an optical detector, the optical detector being configured to detect detection light radiated from the biological tissue irradiated with infrared light, the controller being configured to transmit the created image to a display apparatus such that the display apparatus displays the created image,
wherein the controller is configured to analyze the detection result by the optical detector to identify an affected part in the biological tissue, the controller being configured to superimpose information on the affected part on the image of the biological tissue and cause the display apparatus to display the superimposed image, the controller being configured to control an irradiation of the information on the affected part to the biological tissue by a projector configured to irradiate the biological tissue with light.

21. A projection method comprising:
irradiating a biological tissue with infrared light;
detecting detection light radiated from the biological tissue irradiated with the infrared light;
creating an image of the biological tissue using a detection result of the detection light;
displaying the image of the biological tissue on a display apparatus; and
controlling an irradiation of light by a projecting apparatus such that contents of an input are reflected to the biological tissue based on the input to the display apparatus configured to display the image of the biological tissue.

22. A projection method comprising:
irradiating a biological tissue with infrared light;
detecting detection light radiated from the biological tissue irradiated with the infrared light;
creating an image of the biological tissue using a detection result of the detection light;
displaying the image of the biological tissue on a display apparatus;
analyzing the detection result to identify an affected part in the biological tissue, superimposing information on the affected part on the image of the biological tissue and causing the display apparatus to display the superimposed image; and
controlling an irradiation of the information on the affected part to the biological tissue by a projecting apparatus configured to irradiate the biological tissue with light.

23. A projecting apparatus comprising:
a projector configured to irradiate a biological tissue with first light; and
a controller configured to control the irradiation with the first light by the projector such that contents of an input are reflected to the biological tissue based on the input to a display apparatus configured to display an image of the biological tissue, the image being created using a detection result by an optical detector, the optical detector being configured to detect detection light radiated from the biological tissue irradiated with infrared light.

24. A projecting apparatus comprising:
a projector configured to irradiate a biological tissue with light; and
a controller configured to analyze a detection result by an optical detector configured to detect detection light radiated from the biological tissue irradiated with infrared light to identify an affected part in the biological tissue, the controller being configured to superimpose information on the affected part on an image of the biological tissue and cause a display apparatus to display the superimposed image, the controller being configured to control an irradiation of the information on the affected part to the biological tissue by the projector.
